SEQUENCE LISTING (59 sequences)

SEQ ID NO: 1 - DNA, 32 bp, synthetic construct
  source 1..32: mol_type = other DNA
  modified_base order(3,5,23): mod_base = OTHER; note = 2-Prime-O-Methoxyethyl modified nucleoside
  modified_base order(6..11,21,30..32): mod_base = OTHER; note = 2-Prime-O-Methyl modified nucleoside
  modified_base order(12..17,22,24..29): mod_base = OTHER; note = 2-Prime-Fluoro modified nucleoside
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = Phosphorothioate linkage
  modified_base 20^21: mod_base = OTHER; note = Methylphosphonate linkage
  modified_base 19: mod_base = OTHER; note = DNA nucleotide carrying a Benner's base
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature order(1..18,21..32): note = RNA
  misc_feature order(19,20): note = DNA
  misc_feature 1..32: note = ALDH2-01/RM4727
ctgtgacagttttcacttnagtgtatgcctgc

SEQ ID NO: 2 - DNA, 32 bp, synthetic construct; identical to SEQ ID NO: 1
  source 1..32: mol_type = other DNA
  modified_base order(3,5,23): mod_base = OTHER; note = 2-Prime-O-Methoxyethyl modified nucleoside
  modified_base order(6..12,14,16,21,25,27,29..32): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = Phosphorothioate linkage
  modified_base 20^21: mod_base = OTHER; note = Methylphosphonate linkage
  modified_base 19: mod_base = OTHER; note = DNA nucleotide carrying a Benner's base
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature order(1..18,21..32): note = RNA
  misc_feature order(19,20): note = DNA
  misc_feature 1..32: note = ALDH2-02/RM4728

SEQ ID NO: 3 - DNA, 32 bp, synthetic construct
  source 1..32: mol_type = other DNA
  modified_base order(3,5,23): mod_base = OTHER; note = 2-Prime-O-Methoxyethyl modified nucleoside
  modified_base order(6..11,21,30..32): mod_base = OTHER; note = 2-Prime-O-Methyl modified nucleoside
  modified_base order(12..17,22,24..29): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = Phosphorothioate linkage
  modified_base 20^21: mod_base = OTHER; note = Methylphosphonate linkage
  misc_feature order(1..19,21..32): note = RNA
  misc_feature 20: note = DNA
  misc_feature 1..32: note = ALDH2-03
  modified_base order(2,4,18): mod_base = OTHER; note = 2-Prime-O-Methoxyethyl modified 5-methyl Uridine
  modified_base 1: mod_base = OTHER; note = 2-Prime-O-Methoxyethyl modified 5-methyl Cytidine
ctgtgacagttttcacttcagtgtatgcctgc

SEQ ID NO: 4 - DNA, 32 bp, synthetic construct; identical to SEQ ID NO: 3
  source 1..32: mol_type = other DNA
  modified_base order(3,5,23): mod_base = OTHER; note = 2-Prime-O-Methoxyethyl modified nucleoside
  modified_base order(6..12,14,16,21,25,27,29..32): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = Phosphorothioate linkage
  modified_base 20^21: mod_base = OTHER; note = Methylphosphonate linkage
  misc_feature 1..32: note = ALDH2-04
  misc_feature order(1..19,21..32): note = RNA
  misc_feature 20: note = DNA
  modified_base order(2,4,18): mod_base = OTHER; note = 2-Prime-O-Methoxyethyl modified 5-methyl Uridine
  modified_base 1: mod_base = OTHER; note = 2-Prime-O-Methoxyethyl modified 5-methyl Cytidine

SEQ ID NO: 5 - DNA, 32 bp, synthetic construct; identical to SEQ ID NO: 1
  source 1..32: mol_type = other DNA
  modified_base order(3,5,23): mod_base = OTHER; note = 2-Prime-O-Methoxyethyl modified nucleoside
  modified_base order(6..11,21,30..32): mod_base = OTHER; note = 2-Prime-O-Methyl modified nucleoside
  modified_base order(12..17,22,24..29): mod_base = OTHER; note = 2-Prime-Fluoro modified nucleoside
  modified_base order(1^2,31^32): mod_base = OTHER; note = (1,3-dimethylimidazolidin-2-ylidene) phosphoramidate linkage
  modified_base order(6..20,21..31): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..32: note = ALDH2-05/RM4729
  misc_feature order(1..18,21..32): note = RNA
  misc_feature order(19,20): note = DNA

SEQ ID NO: 6 - DNA, 32 bp, synthetic construct; identical to SEQ ID NO: 1
  source 1..32: mol_type = other DNA
  modified_base order(3,5,23): mod_base = OTHER; note = 2-Prime-O-Methoxyethyl modified nucleoside
  modified_base order(6..12,14,16,21,25,27,29..32): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base order(1^2,31^32): mod_base = OTHER; note = (1,3-dimethylimidazolidin-2-ylidene) phosphoramidate linkage
  modified_base order(6..20,21..31): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..32: note = ALDH2-06/RM4730
  misc_feature order(19,20): note = DNA
  misc_feature order(1..18,21..32): note = RNA

SEQ ID NO: 7 - DNA, 32 bp, synthetic construct; identical to SEQ ID NO: 3
  source 1..32: mol_type = other DNA
  modified_base order(3,5,23): mod_base = OTHER; note = 2-Prime-O-Methoxyethyl modified nucleoside
  modified_base order(6..11,21,30..32): mod_base = OTHER; note = 2-Prime-O-Methyl modified nucleoside
  modified_base order(12..17,22,24..29): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,31^32): mod_base = OTHER; note = (1,3-dimethylimidazolidin-2-ylidene) phosphoramidate linkage
  modified_base order(6..20,21..31): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..32: note = ALDH2-07
  misc_feature order(1..19,21..32): note = RNA
  misc_feature 20: note = DNA

SEQ ID NO: 8 - DNA, 32 bp, synthetic construct; identical to SEQ ID NO: 3
  source 1..32: mol_type = other DNA
  modified_base order(3,5,23): mod_base = OTHER; note = 2-Prime-O-Methoxyethyl modified nucleoside
  modified_base order(6..12,14,16,21,25,27,29..32): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,31^32): mod_base = OTHER; note = (1,3-dimethylimidazolidin-2-ylidene) phosphoramidate linkage
  modified_base order(6..20,21..31): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..32: note = ALDH2-08
  misc_feature order(1..19,21..32): note = RNA
  misc_feature 20: note = DNA

SEQ ID NO: 9 - DNA, 30 bp, synthetic construct
  source 1..30: mol_type = other DNA
  modified_base order(1,3,4,21): mod_base = OTHER; note = 2-Prime-O-Methoxyethyl modified nucleoside
  modified_base order(6..9,19,28..30): mod_base = OTHER; note = 2-Prime-O-Methyl modified nucleoside
  modified_base order(10..15,20,22..27): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  modified_base 17: mod_base = OTHER; note = DNA nucleotide carrying a Benner's base
  misc_feature 1..30: note = ALDH2-09/RM4731
  misc_feature order(1..16,19..30): note = RNA
  misc_feature order(17,18): note = DNA
gtgacagttttcacttnagtgtatgcctgc

SEQ ID NO: 10 - DNA, 30 bp, synthetic construct; identical to SEQ ID NO: 9
  source 1..30: mol_type = other DNA
  modified_base order(1,3,4,21): mod_base = OTHER; note = 2-Prime-O-Methoxyethyl modified nucleoside
  modified_base order(6..10,12,14,19,23,25,27..30): mod_base = OTHER; note = 2-Prime-O-Methyl modified nucleoside
  modified_base order(11,13,15,20,22,24,26): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  modified_base 17: mod_base = OTHER; note = DNA nucleotide carrying a Benner's base
  misc_feature 1..30: note = ALDH2-10/RM4732
  misc_feature order(17,18): note = DNA
  misc_feature order(1..16,19..30): note = RNA

SEQ ID NO: 11 - DNA, 30 bp, synthetic construct
  source 1..30: mol_type = other DNA
  modified_base order(1,3,4,21): mod_base = OTHER; note = 2-Prime-O-Methoxyethyl modified nucleoside
  modified_base order(6..9,19,28..30): mod_base = OTHER; note = 2-Prime-O-Methyl modified nucleoside
  modified_base order(10..15,20,22..27): mod_base = OTHER; note = 2-Prime-Fluoro modified nucleoside
  modified_base 17: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  misc_feature 1..30: note = ALDH2-11
  misc_feature order(1..17,19..30): note = RNA
  misc_feature 18: note = DNA
gtgacagttttcacttcagtgtatgcctgc

SEQ ID NO: 12 - DNA, 30 bp, synthetic construct; identical to SEQ ID NO: 11
  source 1..30: mol_type = other DNA
  modified_base order(1,3,4,21): mod_base = OTHER; note = 2-Prime-O-Methoxyethyl modified nucleoside
  modified_base order(6..10,12,14,19,23,25,27..30): mod_base = OTHER; note = 2-Prime-O-Methyl modified nucleoside
  modified_base order(11,13,15,20,22,24,26): mod_base = OTHER; note = 2-Prime-Fluoro modified nucleoside
  modified_base 17: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  misc_feature 1..30: note = ALDH2-12
  misc_feature order(1..17,19..30): note = RNA
  misc_feature 18: note = DNA

SEQ ID NO: 13 - DNA, 30 bp, synthetic construct; identical to SEQ ID NO: 9
  source 1..30: mol_type = other DNA
  modified_base order(1,3,4,21): mod_base = OTHER; note = 2-Prime-O-Methoxyethyl modified nucleoside
  modified_base order(6..9,19,28..30): mod_base = OTHER; note = 2-Prime-O-Methyl modified nucleoside
  modified_base order(10..15,20,22..27): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..18,19..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  modified_base 17: mod_base = OTHER; note = DNA nucleotide carrying a Benner's base
  misc_feature order(1..16,19..30): note = RNA
  misc_feature order(17,18): note = DNA
  misc_feature 1..30: note = ALDH2-13/RM4733

SEQ ID NO: 14 - DNA, 30 bp, synthetic construct; identical to SEQ ID NO: 9
  source 1..30: mol_type = other DNA
  modified_base order(1,3,4,21): mod_base = OTHER; note = 2-Prime-O-Methoxyethyl modified nucleoside
  modified_base order(6..10,12,14,19,23,25,27..30): mod_base = OTHER; note = 2-Prime-O-Methyl modified nucleoside
  modified_base order(11,13,15,20,22,24,26): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..18,19..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  modified_base 17: mod_base = OTHER; note = DNA nucleotide carrying a Benner's base
  misc_feature 1..30: note = ALDH2-14/RM4734
  misc_feature order(1..16,19..30): note = RNA
  misc_feature order(17,18): note = DNA

SEQ ID NO: 15 - DNA, 30 bp, synthetic construct; identical to SEQ ID NO: 11
  source 1..30: mol_type = other DNA
  modified_base order(1,3,4,21): mod_base = OTHER; note = 2-Prime-O-Methoxyethyl modified nucleoside
  modified_base order(6..9,19,28..30): mod_base = OTHER; note = 2-Prime-O-Methyl modified nucleoside
  modified_base order(10..15,20,22..27): mod_base = OTHER; note = 2-Prime-Fluoro modified nucleoside
  modified_base 17: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..18,19..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  misc_feature 1..30: note = ALDH2-15
  misc_feature order(1..17,19..30): note = RNA
  misc_feature 18: note = DNA

SEQ ID NO: 16 - DNA, 30 bp, synthetic construct; identical to SEQ ID NO: 11
  source 1..30: mol_type = other DNA
  modified_base order(1,3,4,21): mod_base = OTHER; note = 2-Prime-O-Methoxyethyl modified nucleoside
  modified_base order(6..10,12,14,19,23,25,27..30): mod_base = OTHER; note = 2-Prime-O-Methyl modified nucleoside
  modified_base order(11,13,15,20,22,24,26): mod_base = OTHER; note = 2-Prime-Fluoro modified nucleoside
  modified_base 17: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..18,19..29): mod_base = OTHER; note = Phosphorothioate linkage
  modified_base 18^19: mod_base = OTHER; note = Methylphosphonate linkage
  modified_base order(2,16): mod_base = OTHER; note = 2-Prime-O-Methoxyethyl modified 5-methyl Uridine
  modified_base 5: mod_base = OTHER; note = 2-Prime-O-Methoxyethyl modified 5-methyl Cytidine
  misc_feature 1..30: note = ALDH2-16
  misc_feature order(1..17,19..30): note = RNA
  misc_feature 18: note = DNA

SEQ ID NO: 17 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..7,17,26..28): mod_base = OTHER; note = 2-Prime-O-Methyl modified nucleoside
  modified_base order(8..13,18,20..25): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  modified_base 15: mod_base = OTHER; note = DNA nucleotide carrying a Benner's base
  misc_feature 1..28: note = ALDH2-17/RM4735
  misc_feature order(1..14,17..28): note = RNA
  misc_feature order(15,16): note = DNA
gacagttttcacttnagtgtatgcctgc

SEQ ID NO: 18 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..8,10,12,17,21,23,25..28): mod_base = OTHER; note = 2-Prime-O-Methyl modified nucleoside
  modified_base order(9,11,13,18,20,22,24): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  modified_base 15: mod_base = OTHER; note = DNA nucleotide carrying a Benner's base
  misc_feature 1..28: note = ALDH2-18/RM4736
  misc_feature order(1..14,17..28): note = RNA
  misc_feature order(15,16): note = DNA
gacagttttcacttnagtgtatgcctgc

SEQ ID NO: 19 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..7,17,26..28): mod_base = OTHER; note = 2-Prime-O-Methyl modified nucleoside
  modified_base order(8..13,18,20..25): mod_base = OTHER; note = 2-Prime-Fluoro modified nucleoside
  modified_base 15: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  misc_feature 1..28: note = ALDH2-19
  misc_feature order(1..15,17..28): note = RNA
  misc_feature 16: note = DNA
gacagttttcacttcagtgtatgcctgc

SEQ ID NO: 20 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..8,10,12,17,21,23,25..28): mod_base = OTHER; note = 2-Prime-O-Methyl modified nucleoside
  modified_base order(9,11,13,18,20,22,24): mod_base = OTHER; note = 2-Prime-Fluoro modified nucleoside
  modified_base 15: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  misc_feature 1..28: note = ALDH2-20
  misc_feature order(1..15,17..28): note = RNA
  misc_feature 16: note = DNA
gacagttttcacttcagtgtatgcctgc

SEQ ID NO: 21 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..7,17,26..28): mod_base = OTHER; note = 2-Prime-O-Methyl modified nucleoside
  modified_base order(8..13,18,20..25): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..16,17..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  modified_base 15: mod_base = OTHER; note = DNA nucleotide carrying a Benner's base
  misc_feature 1..28: note = ALDH2-21/RM4737
  misc_feature order(1..14,17..28): note = RNA
  misc_feature order(15,16): note = DNA
gacagttttcacttnagtgtatgcctgc

SEQ ID NO: 22 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..8,10,12,17,21,23,25..28): mod_base = OTHER; note = 2-Prime-O-Methyl modified nucleoside
  modified_base order(9,11,13,18,20,22,24): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..16,17..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  modified_base 15: mod_base = OTHER; note = DNA nucleotide carrying a Benner's base
  misc_feature 1..28: note = ALDH2-22/RM4738
  misc_feature order(1..14,17..28): note = RNA
  misc_feature order(15,16): note = DNA
gacagttttcacttnagtgtatgcctgc

SEQ ID NO: 23 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..7,17,26..28): mod_base = OTHER; note = 2-Prime-O-Methyl modified nucleoside
  modified_base order(8..13,18,20..25): mod_base = OTHER; note = 2-Prime-Fluoro modified nucleoside
  modified_base 15: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..16,17..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  misc_feature 1..28: note = ALDH2-23
  misc_feature order(1..15,17..28): note = RNA
  misc_feature 16: note = DNA
gacagttttcacttcagtgtatgcctgc

SEQ ID NO: 24 - DNA, 28 bp, synthetic construct
  source 1..28: mol_type = other DNA
  modified_base order(1,2,4,5,19): mod_base = OTHER; note = 2-Prime-O-Methoxyethyl modified nucleoside
  modified_base order(6..8,10,12,17,21,23,25..28): mod_base = OTHER; note = 2-Prime-O-Methyl modified nucleoside
  modified_base order(9,11,13,18,20,22,24): mod_base = OTHER; note = 2-Prime-Fluoro modified nucleoside
  modified_base 15: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..16,17..27): mod_base = OTHER; note = Phosphorothioate linkage
  modified_base 16^17: mod_base = OTHER; note = Methylphosphonate linkage
  modified_base 3: mod_base = OTHER; note = 2-Prime-O-Methoxyethyl modified 5-methyl Cytidine
  modified_base 14: mod_base = OTHER; note = 2-Prime-O-Methoxyethyl modified 5-methyl Uridine
  misc_feature 1..28: note = ALDH2-24
  misc_feature order(1..15,17..28): note = RNA
  misc_feature 16: note = DNA
gacagttttcacttcagtgtatgcctgc

SEQ ID NO: 25 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..5,15,24..26): mod_base = OTHER; note = 2-Prime-O-Methyl modified nucleoside
  modified_base order(6..11,16,18..23): mod_base = OTHER; note = 2-Prime-Fluoro modified nucleoside
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a Benner's base
  misc_feature 1..26: note = ALDH2-25/RM4739
  misc_feature order(1..12,15..26): note = RNA
  misc_feature order(13,14): note = DNA
cagttttcacttnagtgtatgcctgc

SEQ ID NO: 26 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..6,8,10,15,19,21,23..26): mod_base = OTHER; note = 2-Prime-O-Methyl modified nucleoside
  modified_base order(7,9,11,16,18,20,22): mod_base = OTHER; note = 2-Prime-Fluoro modified nucleoside
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a Benner's base
  misc_feature 1..26: note = ALDH2-26/RM4740
  misc_feature order(1..12,15..26): note = RNA
  misc_feature order(13,14): note = DNA
cagttttcacttnagtgtatgcctgc

SEQ ID NO: 27 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..5,15,24..26): mod_base = OTHER; note = 2-Prime-O-Methyl modified nucleoside
  modified_base order(6..11,16,18..23): mod_base = OTHER; note = 2-Prime-Fluoro modified nucleoside
  modified_base 13: mod_base = OTHER; note = 2-Prime-2-Prime-Difluoro modified nucleoside
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  misc_feature 1..26: note = ALDH2-27
  misc_feature order(1..13,15..26): note = RNA
  misc_feature 14: note = DNA
cagttttcacttcagtgtatgcctgc

SEQ ID NO: 28 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..6,8,10,15,19,21,23..26): mod_base = OTHER; note = 2-Prime-O-Methyl modified nucleoside
  modified_base order(7,9,11,16,18,20,22): mod_base = OTHER; note = 2-Prime-Fluoro modified nucleoside
  modified_base 13: mod_base = OTHER; note = 2-Prime-2-Prime-Difluoro modified nucleoside
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  misc_feature 1..26: note = ALDH2-28
  misc_feature order(1..13,15..26): note = RNA
  misc_feature 14: note = DNA
cagttttcacttcagtgtatgcctgc

SEQ ID NO: 29 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..5,15,24..26): mod_base = OTHER; note = 2-Prime-O-Methyl modified nucleoside
  modified_base order(6..11,16,18..23): mod_base = OTHER; note = 2-Prime-Fluoro modified nucleoside
  modified_base order(1^2,25^26): mod_base = OTHER; note = (1,3-dimethylimidazolidin-2-ylidene) phosphoramidate linkage
  modified_base order(4..14,15..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a Benner's base
  misc_feature 1..26: note = ALDH2-29/RM4741
  misc_feature order(1..12,15..26): note = RNA
  misc_feature order(13,14): note = DNA
cagttttcacttnagtgtatgcctgc

SEQ ID NO: 30 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..6,8,10,15,19,21,23..26): mod_base = OTHER; note = 2-Prime-O-Methyl modified nucleoside
  modified_base order(7,9,11,16,18,20,22): mod_base = OTHER; note = 2-Prime-Fluoro modified nucleoside
  modified_base order(1^2,25^26): mod_base = OTHER; note = (1,3-dimethylimidazolidin-2-ylidene) phosphoramidate linkage
  modified_base order(4..14,15..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a Benner's base
  misc_feature 1..26: note = ALDH2-30/RM4742
  misc_feature order(1..12,15..26): note = RNA
  misc_feature order(13,14): note = DNA
cagttttcacttnagtgtatgcctgc

SEQ ID NO: 31 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..5,15,24..26): mod_base = OTHER; note = 2-Prime-O-Methyl modified nucleoside
  modified_base order(6..11,16,18..23): mod_base = OTHER; note = 2-Prime-Fluoro modified nucleoside
  modified_base 13: mod_base = OTHER; note = 2-Prime-2-Prime-Difluoro modified nucleoside
  modified_base order(1^2,25^26): mod_base = OTHER; note = (1,3-dimethylimidazolidin-2-ylidene) phosphoramidate linkage
  modified_base order(4..14,15..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  misc_feature 1..26: note = ALDH2-31
  misc_feature order(1..13,15..26): note = RNA
  misc_feature 14: note = DNA
cagttttcacttcagtgtatgcctgc

SEQ ID NO: 32 - DNA, 26 bp, synthetic construct
  source 1..26: mol_type = other DNA
  modified_base order(2,3,17): mod_base = OTHER; note = 2-Prime-O-Methoxyethyl modified nucleoside
  modified_base order(4..6,8,10,15,19,21,23..26): mod_base = OTHER; note = 2-Prime-O-Methyl modified nucleoside
  modified_base order(7,9,11,16,18,20,22): mod_base = OTHER; note = 2-Prime-Fluoro modified nucleoside
  modified_base 13: mod_base = OTHER; note = 2-Prime-2-Prime-Difluoro modified nucleoside
  modified_base order(1^2,25^26): mod_base = OTHER; note = (1,3-dimethylimidazolidin-2-ylidene) phosphoramidate linkage
  modified_base order(4..14,15..25): mod_base = OTHER; note = Phosphorothioate linkage
  modified_base 14^15: mod_base = OTHER; note = Methylphosphonate linkage
  modified_base 1: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  misc_feature 1..26: note = ALDH2-32
  misc_feature order(1..13,15..26): note = RNA
  misc_feature 14: note = DNA
cagttttcacttcagtgtatgcctgc

SEQ ID NO: 33 - DNA, 30 bp, synthetic construct
  source 1..30: mol_type = other DNA
  modified_base order(3,5,23): mod_base = OTHER; note = 2-Prime-O-Methoxyethyl modified nucleoside
  modified_base order(6..11,21,30): mod_base = OTHER; note = 2-Prime-O-Methyl modified nucleoside
  modified_base order(12..17,22,24..29): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..30: note = ALDH2-33/RM4743
  misc_feature order(1..18,21..30): note = RNA
  misc_feature order(19,20): note = DNA
ctgtgacagttttcacttnagtgtatgcct

SEQ ID NO: 34 - DNA, 30 bp, synthetic construct; identical to SEQ ID NO: 33
  source 1..30: mol_type = other DNA
  modified_base order(3,5,23): mod_base = OTHER; note = 2-Prime-O-Methoxyethyl modified nucleoside
  modified_base order(6..12,14,16,21,25,27,29..30): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..30: note = ALDH2-34/RM4744
  misc_feature order(1..18,21..30): note = RNA
  misc_feature order(19,20): note = DNA

SEQ ID NO: 35 - DNA, 30 bp, synthetic construct
  source 1..30: mol_type = other DNA
  modified_base order(3,5,23): mod_base = OTHER; note = 2-Prime-O-Methoxyethyl modified nucleoside
  modified_base order(6..11,21,30): mod_base = OTHER; note = 2-Prime-O-Methyl modified nucleoside
  modified_base order(12..17,22,24..29): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..30: note = ALDH2-35
  misc_feature order(1..19,21..30): note = RNA
  misc_feature 20: note = DNA
ctgtgacagttttcacttcagtgtatgcct

SEQ ID NO: 36 - DNA, 30 bp, synthetic construct; identical to SEQ ID NO: 35
  source 1..30: mol_type = other DNA
  modified_base order(3,5,23): mod_base = OTHER; note = 2-Prime-O-Methoxyethyl modified nucleoside
  modified_base order(6..12,14,16,21,25,27,29..30): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..30: note = ALDH2-36
  misc_feature order(1..19,21..30): note = RNA
  misc_feature 20: note = DNA

SEQ ID NO: 37 - DNA, 30 bp, synthetic construct; identical to SEQ ID NO: 33
  source 1..30: mol_type = other DNA
  modified_base order(3,5,23): mod_base = OTHER; note = 2-Prime-O-Methoxyethyl modified nucleoside
  modified_base order(6..11,21,30): mod_base = OTHER; note = 2-Prime-O-Methyl modified nucleoside
  modified_base order(12..17,22,24..29): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..20,21..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..30: note = ALDH2-37/RM4745
  misc_feature order(1..18,21..30): note = RNA
  misc_feature order(19,20): note = DNA

SEQ ID NO: 38 - DNA, 30 bp, synthetic construct; identical to SEQ ID NO: 33
  source 1..30: mol_type = other DNA
  modified_base order(3,5,23): mod_base = OTHER; note = 2-Prime-O-Methoxyethyl modified nucleoside
  modified_base order(6..12,14,16,21,25,27,29..30): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base order(1^2,29^30): mod_base = OTHER; note = (1,3-dimethylimidazolidin-2-ylidene) phosphoramidate linkage
  modified_base order(6..20,21..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..30: note = ALDH2-38/RM4746
  misc_feature order(1..18,21..30): note = RNA
  misc_feature order(19,20): note = DNA

SEQ ID NO: 39 - DNA, 30 bp, synthetic construct; identical to SEQ ID NO: 35
  source 1..30: mol_type = other DNA
  modified_base order(3,5,23): mod_base = OTHER; note = 2-Prime-O-Methoxyethyl modified nucleoside
  modified_base order(6..11,21,30): mod_base = OTHER; note = 2-Prime-O-Methyl modified nucleoside
  modified_base order(12..17,22,24..29): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..20,21..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..30: note = ALDH2-39
  misc_feature order(1..19,21..30): note = RNA
  misc_feature 20: note = DNA

SEQ ID NO: 40 - DNA, 30 bp, synthetic construct; identical to SEQ ID NO: 35
  source 1..30: mol_type = other DNA
  modified_base order(3,5,23): mod_base = OTHER; note = 2-Prime-O-Methoxyethyl modified nucleoside
  modified_base order(6..12,14,16,21,25,27,29..30): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,29^30): mod_base = OTHER; note = (1,3-dimethylimidazolidin-2-ylidene) phosphoramidate linkage
  modified_base order(6..20,21..29): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..30: note = ALDH2-40
  misc_feature order(1..19,21..30): note = RNA
  misc_feature 20: note = DNA

SEQ ID NO: 41 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..11,21): mod_base = OTHER; note = 2-Prime-O-Methyl modified nucleoside
  modified_base order(12..17,22,24..28): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..28: note = ALDH2-41/RM4747
  misc_feature order(1..18,21..28): note = RNA
  misc_feature order(19,20): note = DNA
ctgtgacagttttcacttnagtgtatgc

SEQ ID NO: 42 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..12,14,16,21,25,27): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..28: note = ALDH2-42/RM4748
  misc_feature order(1..18,21..28): note = RNA
  misc_feature order(19,20): note = DNA
ctgtgacagttttcacttnagtgtatgc

SEQ ID NO: 43 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..11,21): mod_base = OTHER; note = 2-Prime-O-Methyl modified nucleoside
  modified_base order(12..17,22,24..28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..28: note = ALDH2-43
  misc_feature order(1..19,21..28): note = RNA
  misc_feature 20: note = DNA
ctgtgacagttttcacttcagtgtatgc

SEQ ID NO: 44 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..12,14,16,21,25,27): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..28: note = ALDH2-44
  misc_feature order(1..19,21..28): note = RNA
  misc_feature 20: note = DNA
ctgtgacagttttcacttcagtgtatgc

SEQ ID NO: 45 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..11,21): mod_base = OTHER; note = 2-Prime-O-Methyl modified nucleoside
  modified_base order(12..17,22,24..28): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..20,21..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..28: note = ALDH2-45/RM4749
  misc_feature order(1..18,21..28): note = RNA
  misc_feature order(19,20): note = DNA
ctgtgacagttttcacttnagtgtatgc

SEQ ID NO: 46 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..12,14,16,21,25,27): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base order(1^2,27^28): mod_base = OTHER; note = (1,3-dimethylimidazolidin-2-ylidene) phosphoramidate linkage
  modified_base order(6..20,21..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  modified_base 19: mod_base = OTHER; note = DNA nucleotide carrying a Benner's base
  misc_feature 1..28: note = ALDH2-46/RM4750
  misc_feature order(1..18,21..28): note = RNA
  misc_feature order(19,20): note = DNA
ctgtgacagttttcacttnagtgtatgc

SEQ ID NO: 47 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..11,21): mod_base = OTHER; note = 2-Prime-O-Methyl modified nucleoside
  modified_base order(12..17,22,24..28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..20,21..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..28: note = ALDH2-47
  misc_feature order(1..19,21..28): note = RNA
  misc_feature 20: note = DNA
ctgtgacagttttcacttcagtgtatgc

SEQ ID NO: 48 - DNA, 28 bp, synthetic construct
  source 1..28: mol_type = other DNA
  modified_base order(3,5,23): mod_base = OTHER; note = 2-Prime-O-Methoxyethyl modified nucleoside
  modified_base order(6..12,14,16,21,25,27): mod_base = OTHER; note = 2-Prime-O-Methyl modified nucleoside
  modified_base order(13,15,17,22,24,26,28): mod_base = OTHER; note = 2-Prime-Fluoro modified nucleoside
  modified_base 19: mod_base = OTHER; note = 2-Prime-2-Prime-Difluoro modified nucleoside
  modified_base order(1^2,27^28): mod_base = OTHER; note = (1,3-dimethylimidazolidin-2-ylidene) phosphoramidate linkage
  modified_base order(6..20,21..27): mod_base = OTHER; note = Phosphorothioate linkage
  modified_base 20^21: mod_base = OTHER; note = Methylphosphonate linkage
  modified_base 1: mod_base = OTHER; note = 2-Prime-O-Methoxyethyl modified 5-methyl Cytidine
  modified_base order(2,4,18): mod_base = OTHER; note = 2-Prime-O-Methoxyethyl modified 5-methyl Uridine
  misc_feature 1..28: note = ALDH2-48
  misc_feature order(1..19,21..28): note = RNA
  misc_feature 20: note = DNA
ctgtgacagttttcacttcagtgtatgc

SEQ ID NO: 49 - DNA, 30 bp, synthetic construct
  source 1..30: mol_type = other DNA
  modified_base order(1..15,24): mod_base = OTHER; note = 2-Prime-Fluoro modified nucleoside
  modified_base order(16..23,27..30): mod_base = OTHER; note = 2-Prime-O-Methyl modified nucleoside
  modified_base order(1^2,16^17,26^27,29^30): mod_base = OTHER; note = (1,3-dimethylimidazolidin-2-ylidene) phosphoramidate linkage
  modified_base order(2..16,17..26,27..29): mod_base = OTHER; note = Phosphorothioate linkage
  misc_feature 1..30: note = ALDH2-49/RM4751
  misc_feature order(1..24,27..30): note = RNA
  misc_feature order(25,26): note = DNA
actttgactgtgacagttttcactcagtgt

SEQ ID NO: 50 - DNA, 30 bp, synthetic construct
  source 1..30: mol_type = other DNA
  modified_base order(1..15,24): mod_base = OTHER; note = 2-Prime-Fluoro modified nucleoside
  modified_base order(16..23,27..30): mod_base = OTHER; note = 2-Prime-O-Methyl modified nucleoside
  modified_base order(1^2,16^17,26^27,29^30): mod_base = OTHER; note = (1,3-dimethylimidazolidin-2-ylidene) phosphoramidate linkage
  modified_base order(2..16,17..26,27..29): mod_base = OTHER; note = Phosphorothioate linkage
  modified_base 25: mod_base = OTHER; note = DNA nucleotide carrying a Benner's base
  misc_feature 1..30: note = ALDH2-50/RM4752
  misc_feature order(1..24,27..30): note = RNA
  misc_feature order(25,26): note = DNA
actttgactgtgacagttttcactnagtgt

SEQ ID NO: 51 - DNA, 30 bp, synthetic construct; identical to SEQ ID NO: 49
  source 1..30: mol_type = other DNA
  modified_base order(1..15,24): mod_base = OTHER; note = 2-Prime-Fluoro modified nucleoside
  modified_base order(16..23,27..30): mod_base = OTHER; note = 2-Prime-O-Methyl modified nucleoside
  modified_base 25: mod_base = OTHER; note = 2-Prime-2-Prime-Difluoro modified nucleoside
  modified_base order(1^2,16^17,26^27,29^30): mod_base = OTHER; note = (1,3-dimethylimidazolidin-2-ylidene) phosphoramidate linkage
  modified_base order(2..16,17..26,27..29): mod_base = OTHER; note = Phosphorothioate linkage
  misc_feature 1..30: note = ALDH2-51/RM4753
  misc_feature order(1..25,27..30): note = RNA
  misc_feature 26: note = DNA

SEQ ID NO: 52 - RNA, 39 nt, Homo sapiens
  source 1..39: mol_type = mRNA
gcaggcatacactaaagtgaaaactgtcacagtcaaagt

SEQ ID NO: 53 - DNA, 20 bp, synthetic construct
  source 1..20: mol_type = other DNA
ctcgacgcaagccataacac

SEQ ID NO: 54 - DNA, 20 bp, synthetic construct
  source 1..20: mol_type = other DNA
tggaccgactggaaacgtag

SEQ ID NO: 55 - RNA, 274 nt, Homo sapiens
  source 1..274: mol_type = mRNA
tctggctcgacgcaagccataacactaatacgactcactatagggggagcccagtcaccctttggtggctacaagatgtcggggagtggccgggagttgggcgagtacgggctgcaggcatacactaaagtgaaaactgtcacagtcaaagtgcctcagaagaactcataagaatcatgcaagcttcctccctcagccattgatggaaagttcagcaagatcagcaacaaaaccaagaaaaatgatccttacgtttccagtcggtccacgtttg

SEQ ID NO: 56 - DNA, 20 bp, synthetic construct
  source 1..20: mol_type = other DNA
tggtggctacaagatgtcgg

SEQ ID NO: 57 - DNA, 22 bp, synthetic construct
  source 1..22: mol_type = other DNA
ttatgagttcttctgaggcact

SEQ ID NO: 58 - DNA, 24 bp, synthetic construct
  source 1..24: mol_type = other DNA
  modified_base order(2,7,14..16): mod_base = OTHER; note = Locked Nucleic Acid
acagttttcacttcagtgtatgcc

SEQ ID NO: 59 - DNA, 25 bp, synthetic construct
  source 1..25: mol_type = other DNA
  modified_base order(2,7,14..16): mod_base = OTHER; note = Locked Nucleic Acid
acagttttcactttagtgtatgccc

(19) United States
(12) Patent Application Publication
(10) Pub. No.: US 2026/0234633 A1
(43) Pub. Date: Aug. 13, 2026

(54) ANTISENSE OLIGONUCLEOTIDES FOR THE TREATMENT OF ALDEHYDE DEHYDROGENASE 2 DEFICIENCY

(71) Applicant: ProQR Therapeutics II B.V., Leiden (NL)

(72) Inventors: Marko POTMAN, Leiden (NL); Aliye Seda YILMAZ-ELIS, Leiden (NL); Gerardus Johannes PLATENBURG, Leiden (NL); Daniel Anton DE BOER, Leiden (NL)

(73) Assignee: ProQR Therapeutics II B.V., Leiden (NL)

(21) Appl. No.: 19/134,547

(22) PCT Filed: Nov. 30, 2023
(86) PCT No.: PCT/EP2023/083678

§ 371 Date: May 30, 2025

(30) Foreign Application Priority Data
Dec. 1, 2022 (GB) 2218090.5

(51) Int. Cl. C12N 15/113; C12N 15/86

(52) U.S. Cl. CPC C12N 15/1137; C12N 15/86; C12Y 102/01003; C12N 2310/11; C12N 2310/3125; C12N 2310/313; C12N 2310/314; C12N 2310/315; C12N 2310/321; C12N 2310/322; C12N 2750/14143

(57) ABSTRACT

The invention relates to the field of diseases caused by alcohol intolerance, such as those that are the result of an ALDH2*2 mutation in the human ALDH2 gene. The invention involves oligonucleotides and the use thereof in RNA editing methods in targeting a target adenosine in an endogenous human ALDH2 transcript in a cell, such as a G>A mutation in the transcript molecule C of a mutant ALDH2 gene coding for a mutant p.E504K ALDH2 protein, especially in the liver.

### TECHNICAL FIELD

This invention relates to the field of medicine. It relates to the field of diseases that are caused by a mutant aldehyde dehydrogenase 2 (ALDH2) protein. The invention involves the use of nucleotide editing technology in targeting the ALDH2 gene transcript to bring about amino acid changes that restore the normal function of the ALDH2 protein in regulating ethanol metabolism.

### BACKGROUND

Mitochondrial aldehyde dehydrogenase 2 (ALDH2) in the liver removes toxic aldehydes including acetaldehyde, an intermediate of ethanol metabolism and is therefore essential for alcohol detoxification. The well-known alcohol-induced flush reaction is caused by a mutation in the structural gene for ALDH2 protein (Yoshida A et al. 1984. Proc Natl Acad Sci USA. 81:258-261). ALDH2 also plays a role in the conversion and removal of 4-hydroxynonenal (HNE), which is a reactive aldehyde. At low levels this compound is beneficial, but at higher levels it is toxic. The variant allele of ALDH2, designated ALDH2*2, encodes a lysine for glutamate substitution at residue 504 of the mature enzyme. The wild-type allele is designated ALDH2*1. The wild-type protein is often referred to as ALDH2E, whereas the mutant protein is often referred to as ALDH2K. Further (other) mutant forms of the allele are known as ALDH2*3, ALDH2*4, and so on (Chen C-H et al. 2020. EBioMedicine 55:102753). About 40% to 45% of East Asians (approximately 8% of the world population) inherit the inactive ALDH2*2 variant and show the characteristic alcohol flush reaction after consuming alcohol.

The alcohol flushing syndrome is not benign. Unequivocal epidemiological data and meta-analysis have consistently shown that alcohol consumption among ALDH2*2 carriers leads to a significantly increased risk for several cancers, in particular, the upper aerodigestive tract cancers. The activity of ALDH2K is partially dominant-negative over that of the wild-type version, due to the structural alterations introduced by the mutation to the ALDH2 tetramer complex. As a result, individuals with a heterozygous genotype have less than half the wild-type activity, and mutant homozygotes have very low residual activity.

Acetaldehyde binds to cellular proteins and DNA, which may lead to DNA damage and organ injury. Specifically, endogenous aldehydes are detrimental to hematopoietic stem cells that are defective in Fanconi anaemia DNA repair, which makes that patients suffering from Fanconi anaemia with the ALDH2*2 allele exhibit accelerated disease progression. Besides that, ALDH2*2 increases the risk for gastrointestinal cancers, such as gastric carcinoma, oesophageal cancer, and colon cancer. Mutant mice carrying the lysine-for-glutamate mutation (equivalent to the human E504K mutation) recapitulate essentially all human phenotypes including impaired clearance of acetaldehyde, increased sensitivity to acute or chronic alcohol-induced toxicity, and reduced ALDH2 expression due to the dominant-negative effect of the mutation. When treated with a chemical carcinogen, the mutant mice exhibited increased DNA damage response in hepatocytes, pronounced liver injury, and accelerated development of hepatocellular carcinoma (Jin S et al. 2015. Proc Natl Acad Sci USA. 112(29):9088-9093), supporting the notion that the common human ALDH2*2 variant is a significant risk factor for hepatocarcinogenesis. Aldehyde toxicity is not limited to cancer; it has also been implicated in many other diseases with increased vulnerability among the ALDH2*2 subject, ranging from osteoporosis, cardiovascular disease, Alzheimer's disease, and rare genetic disease such as Fanconi anaemia mentioned above.

The ALDH2*2 variant has thus far been characterized as an East Asian-specific polymorphism. Extensive global geographic and population mapping based on data of more than 80,000 individuals from 366 population samples has confirmed that the ALDH2*2 allele is highly concentrated in areas of Southeast China, Japan, Korea, Taiwan, Singapore, and Vietnam. In Taiwan the prevalence of the ALDH2*2 carrier is as high as 49%, affecting half of the population of a single country (Luo H R et al. 2009. Gene. 435(1-2):96-103).

A potential therapeutic known as Alda-1 (AD-6626) was developed as a small molecule treatment for alcohol intoxication because the molecule improved outcomes in animal models of myocardial infarction, stroke, radiation dermatitis, and pain. It was thought to create a molecular patch restoring the enzymatic activity of the mutant ALDH2 protein (Chen C-H et al. 2008. Science 321(5895):1493-1495). However, the molecule appears not to be developed further for the treatment of human ALDH2*2 subjects. WO2014/160185 discloses a range of small molecules (like Alda-1) that function as modulators of ALDH2 activity. WO2019/092282, WO2019/143621, WO2020/206350 and WO2022/104366 disclose nucleic acid-based therapeutics that aim to reduce the expression of ALDH2 in subjects that suffer from alcohol use disorder (AUD), which is a disorder that appears less of a risk in ALDH2*2 subjects that generally are not prone to high alcohol intake due to the alcohol intolerance issues that arise from the ALDH2*2 mutation.

A treatment for acute intoxication with ethylene glycol and methanol was approved by the FDA in 1997 for fomepizole (Antizol). However, this product does not target ALDH2 and does not address ethanol intoxication.

The present invention aims to provide one or more alternative, and/or improved, compounds or compositions for use in the treatment of alcohol intolerance due to the ALDH2K mutant protein.

### SUMMARY OF THE INVENTION

Disclosed herein is an RNA editing oligonucleotide (EON) capable of forming a double-stranded complex with a region of an endogenous human ALDH2 transcript molecule in a cell, wherein the region of the ALDH2 transcript molecule comprises a target adenosine, and wherein the double-stranded complex can recruit an endogenous ADAR enzyme to deaminate the target adenosine into an inosine, thereby editing the ALDH2 transcript molecule. Preferably, the ALDH2 transcript molecule is a pre-mRNA or an mRNA molecule. In an embodiment, the cell is a human liver cell, preferably a hepatocyte. A preferred target adenosine is an adenosine resulting from a G>A mutation in the human ALDH2 gene leading to a mutant p.E504K ALDH2 protein. In an embodiment, the EON comprises at least one nucleotide comprising one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution. Disclosed is also a vector, preferably a viral vector, more preferably an adeno-associated virus (AAV) vector, comprising a nucleic acid molecule encoding an EON as disclosed herein. Disclosed is also a pharmaceutical composition comprising an EON as disclosed herein, or a vector as disclosed herein, and a pharmaceutically acceptable carrier.

Disclosed herein is an EON, a vector, or a pharmaceutical composition as disclosed herein, for use in the treatment of a disorder caused by an ALDH2 deficiency, preferably caused by ALDH2*2. Disclosed herein is a use of an EON, or a vector, as disclosed, in the manufacture of a medicament for the treatment of ALDH2*2 induced alcohol intolerance, such as alcohol intoxication, alcohol poisoning, or symptoms of alcohol consumption. A symptom of alcohol consumption is for instance a hangover symptom, such as dehydration, fatigue, headache, body aches, vomiting, diarrhoea, flatulence, weakness, elevated body temperature and heart rate, hypersalivation, difficulty concentrating, sweating, anxiety, dysphoria, irritability, sensitivity to light and noise, erratic motor function, trouble sleeping, severe hunger, halitosis, and lack of depth perception.

Disclosed is a method of treating a disorder caused by an ALDH2 deficiency, preferably caused by ALDH2*2, in a patient in need thereof, the method comprising contacting a ALDH2 polynucleotide in a cell of the subject with an EON capable of effecting an ADAR-mediated adenosine to inosine alteration of an adenosine associated with the ALDH2 deficiency, thereby treating the patient. Disclosed is also a method of treating a disorder caused by ALDH2*2, the method comprising administering to a patient in need thereof a therapeutically effective amount of an EON as disclosed, a vector as disclosed, or a pharmaceutical composition as disclosed. Disclosed is also a method of editing a ALDH2 polynucleotide, the method comprising contacting the ALDH2 polynucleotide with an EON capable of effecting an adenosine deaminase acting on RNA (ADAR)-mediated adenosine to inosine alteration of an adenosine associated with alcohol intolerance, thereby editing the ALDH2 polynucleotide. Preferably, the ALDH2 transcript is from an ALDH2*2 mutated gene. Disclosed is also a method of treating ALDH2*2 induced alcohol intolerance, or a disorder caused by said alcohol intolerance, in a patient in need thereof, the method comprising contacting a ALDH2 polynucleotide in a cell of the subject with an EON capable of effecting an ADAR-mediated adenosine to inosine alteration of an adenosine associated with alcohol intolerance, or a disorder caused by said alcohol intolerance, such as alcohol intoxication, alcohol poisoning, or symptoms of alcohol consumption, thereby treating the patient.

### BRIEF DESCRIPTION OF THE DRAWINGS

One or more embodiments of the invention will now be described, by way of example only, with reference to the accompanying drawings, in which:

FIG. 1 shows on top the human ALDH2 target RNA sequence (5′ to 3′; SEQ ID NO:52) with the target adenosine in bold face and the lysine codon underlined. Below the target sequence, the sequences (also 5′ to 3′) are given of the initial 51 EONs (SEQ ID NO:1 to 51, as indicated) that were initially designed for editing the target adenosine. Some EONs have two names, divided by a backward slash. The chemical modifications in the EONs are as follows: m5Ce is 2′-MOE modified 5-methyl-cytidine; m5Ue is 2′-MOE modified 5-methyl-uridine (identical to a thymidine with a 2′-MOE substitution); Ge and Ae are 2′-MOE modified guanosine and adenosine, respectively; Cm, Am, Um, and Gm are 2′-OMe modified cytidine, adenosine, uridine, and guanosine, respectively; Gf, Cf, Af, and Uf are 2′-F modified guanosine, cytidine, adenosine, and uridine, respectively; Zd (orphan nucleotide) is a deoxynucleotide (deoxycytidine analog) carrying a Benner's base; C2f (orphan nucleotide) is a cytidine with a 2′,2′-difluoro modification; Cd (orphan nucleotide) is a deoxycytidine; Ad is a deoxyadenosine; “!” refers to a PNdmi linkage; “\hat{ }” refers to a MP linkage; “*” refers to a PS linkage. All other internucleoside linkages are phosphodiester linkages.

FIG. 2 shows the percentage editing of the human ALDH2 transcript after gymnotic uptake (GU) of the indicated EONs (black bars) and after co-administration of saponin (grey bars). (A) shows the total percentage including the background signal from the wild type allele. (B) shows the same results after normalization with the non-treated (NT) sample, thereby removing the wild type allele signal.

### DETAILED DESCRIPTION

The inventors of the present invention realized that another approach is possible to target the E504K mutation in ALDH2 to generate a wild type ALDH2 protein and potentially restore proper alcohol metabolism, and thereby to prevent, ameliorate or treat disorders related to accumulation of toxic aldehydes, or alcohol intolerance. This technology is generally referred to as RNA editing. Disclosed herein are oligonucleotides that can be used to specifically deaminate a specific target adenosine in the transcript of the (human) mutant ALDH2 transcript (pre-mRNA and/or mRNA) in vivo, preferably using endogenous deaminating enzymes, to produce an ALDH2 protein that is restored in its function in converting acetaldehyde to acetic acid during ethanol metabolism. By far the most common mutation that is found in the ALDH2 gene is the E504K mutation mentioned above, but the RNA editing technology as disclosed herein is also applicable to other target adenosines within ALDH2 that may be targeted to either restore its function or even to cause a gain-of-function effect. The E504K mutation is a mutation of a GAA codon (coding for glutamate; Glu; E) to an AAA codon (coding for lysine; Lys; K). Specific RNA editing as disclosed herein will convert the first adenosine of the mutant codon to an inosine, which is then read by the translation machinery as a guanosine (AAA>IAA>GAA).

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. As mentioned above, inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. PNAS 92:8298-8302; Montiel-Gonzalez et al. PNAS 2013, 110(45):18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there was a need for methods and means that circumvent such promiscuous editing and only target specific adenosines in a target RNA molecule to become therapeutic applicable. Vogel et al. (2014) showed that such off-target editing can be suppressed by using 2′-O-methyl (2′-OMe) modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area. In one embodiment, one or more mismatching nucleotides, wobbles, or bulges exist between the oligonucleotide and the target sequence. A sole mismatch may be at the site of the nucleoside opposite the target adenosine, but in other embodiments AONs (or RNA editing oligonucleotides, abbreviated to ‘EONs’) were described with multiple bulges and/or wobbles when attached to the target sequence area. It appeared possible to achieve in vitro, ex vivo and in vivo RNA editing with EONs lacking a stem-loop structure and with endogenous ADAR enzymes when the sequence of the EON was carefully selected such that it could attract/recruit ADAR. The ‘orphan nucleoside’, which is defined as the nucleoside in the EON that is positioned directly opposite the target adenosine in the target RNA molecule, did not carry a 2′-OMe modification. The orphan nucleoside can be a deoxyribonucleoside (DNA), wherein the remainder of the EON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the EONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

Disclosed herein are EONs that can produce RNA editing of a target adenosine in the human ALDH2 transcript (pre-mRNA and/or mRNA), through which the resulting ALDH2 protein is restored in its wild-type function: converting acetaldehyde to acetic acid in ethanol metabolism. In a preferred aspect, the EON causes the deamination of the adenosine present at position 1459 of the mutated mRNA, thereby generating an inosine. In other words, the AAA codon encoding lysine (mutant form) at amino acid position 504 is converted to an IAA codon, which is read as GAA that encodes glutamate (wild-type form). In another embodiment, an EON according to the present invention causes the deamination of another adenosine present in the ALDH2 transcript, which may be any adenosine that, when deaminated into an inosine, results in a ALDH2 protein with a wild-type functionality or gain-of-function. Other mutations may be present in the ALDH2 gene (and transcript), that may be targeted through RNA editing thereby restoring the normal ALDH2 function. A preferred mutation that is targeted is the G>A mutation at position 1510 in the transcript, resulting in the p.Glu504Lys ALDH2 protein mutation (c.1510G>A). This protein mutation is often referred to as E487K, but also as E504K, whereas the mutant allele is generally referred to as the ALDH2*2. In literature the mutation is sometimes also referred to as p.Glu487Lys, rs671, c.1510G>A, and p.E504K, although the reference to “c.1459G>A” was not found. Herein, the transcript numbering of the Homo sapiens ALDH2 protein and gene found in ensemble.org is used (transcript ENST00000261733.7), which means that the mutation is referred to as E504K in the protein and c.1510G>A in the transcript.

Although in a preferred embodiment, the EON of the present invention is a single-stranded oligonucleotide comprising an orphan nucleotide opposite the target adenosine, wherein the orphan nucleotide is chemically modified as disclosed herein, and wherein the remainder of the oligonucleotide is chemically modified to prevent it from nuclease breakdown also as disclosed herein, in another embodiment, the invention relates to any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is expressed through a vector, such as an adeno-associated virus (AAV), or wherein the oligonucleotide is in a circular format. It is to be understood that any kind of oligonucleotide-based RNA editing is encompassed by the present invention if it relates to the deamination of a nucleotide in the ALDH2 transcript, preferably the mutation causing E504K, and causes the restoration of the ALDH2 function. In a preferred aspect, the EON of the present invention is a ‘naked’ oligonucleotide, comprising a variety of chemical modifications in the ribose sugar, the base, and/or the internucleoside linkage of one or more of the nucleotides within the sequence, that can hybridize to the ALDH2 transcript or a part thereof that includes the target adenosine, and can recruit endogenous ADAR for the deamination of the target adenosine.

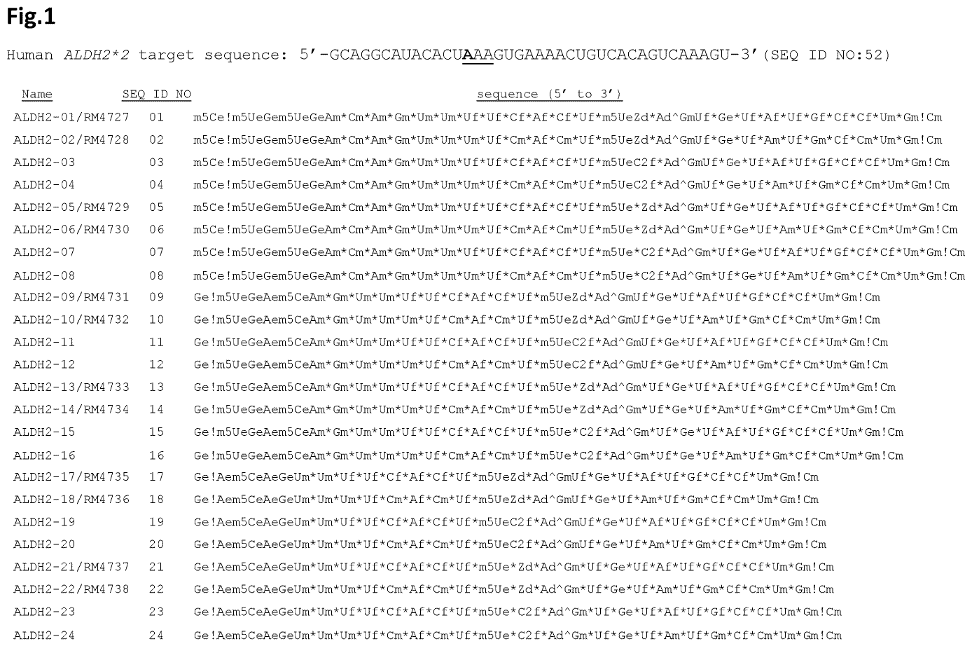

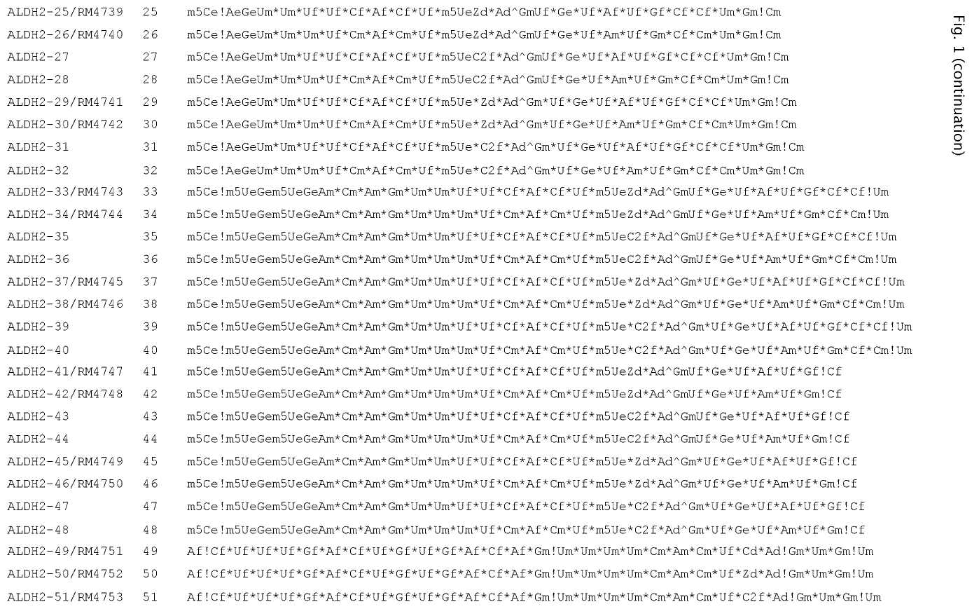

The invention relates to an EON capable of forming a double-stranded complex with a region of an endogenous human ALDH2 transcript molecule in a cell, wherein the region of the ALDH2 transcript molecule comprises a target adenosine, and wherein the double-stranded complex can recruit an endogenous ADAR enzyme to deaminate the target adenosine into an inosine, thereby editing the ALDH2 transcript molecule. The endogenous ADAR enzyme is preferably ADAR2. The cell is preferably a human liver cell, more preferably a human hepatocyte. The ALDH2 transcript molecule is preferably a pre-mRNA or an mRNA molecule. The EON of the present invention preferably targets an adenosine for deamination that causes a dysfunction of the ALDH2 protein. Although several mutations are known that cause a dysfunction of the ALDH2 protein, a preferred mutation that is targeted through the EONs as disclosed herein is the adenosine resulting from a G>A mutation in the ALDH2 gene leading to a mutant p.E504K ALDH2 protein. The EONs as disclosed herein are capable of deaminating the first adenosine in the AAA codon encoding lysine, thereby generating an IAA codon, which is translated to glutamate because the codon is read as GAA. In one embodiment, the EON as disclosed herein comprises or consists of the sequence of any one of the EON sequences depicted in FIG. 1. In an embodiment, the EON of the present invention comprises or is entirely composed of the chemical modifications depicted in FIG. 1.

In one embodiment, an EON according to the invention comprises at least one nucleotide comprising one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution. In an embodiment, the one or more additional modifications in the linkage moiety is each independently selected from a phosphorothioate (PS), phosphonoacetate, phosphorodithioate, methylphosphonate (MP), sulfonylphosphoramidate, or PNdmi internucleotide linkage. Preferably, the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy. In an embodiment, the EON comprises one or more mismatches, wobbles, or bulges, wherein a single mismatch may be present when the target adenosine has an opposite cytidine in the EON. If the orphan nucleotide is a cytidine, that cytidine does not comprise a 2′-OMe ribose substitution.

Disclosed herein is a vector, preferably a viral vector, more preferably an adeno-associated virus (AAV) vector, comprising a nucleic acid molecule encoding an EON as disclosed herein. Further disclosed herein is a pharmaceutical composition comprising an EON as disclosed, or a vector as disclosed, and a pharmaceutically acceptable carrier.

In an embodiment, disclosed is an EON as disclosed herein, a vector as disclosed herein, or a pharmaceutical composition as disclosed herein, for use in the treatment of a disorder caused by an ALDH2 deficiency, preferably caused by ALDH2*2, such as alcohol intoxication, alcohol poisoning, or a symptom of alcohol consumption. In an embodiment, disclosed is an EON as disclosed herein, or a vector as disclosed herein in the manufacture of a medicament for the treatment of ALDH2*2 induced alcohol intolerance, such as alcohol intoxication, alcohol poisoning, or one or more symptoms of alcohol consumption.

In an embodiment, disclosed is a method of editing an ALDH2 polynucleotide, the method comprising contacting the ALDH2 polynucleotide with an EON capable of effecting an adenosine deaminase acting on RNA (ADAR)-mediated adenosine to inosine alteration of an adenosine associated with alcohol intolerance, thereby editing the ALDH2 polynucleotide. In an embodiment, disclosed is a method of treating an ALDH2 deficiency, such as an ALDH2*2 induced alcohol intolerance or intoxication, or a disorder caused by said alcohol intolerance or intoxication, in a patient in need thereof, the method comprising contacting a ALDH2 polynucleotide in a cell of the subject with an EON capable of effecting an ADAR-mediated adenosine to inosine alteration of an adenosine associated with alcohol intolerance, or a disorder caused by said alcohol intolerance, such as alcohol intoxication, alcohol poisoning, or symptoms of alcohol consumption, thereby treating the patient. The preferred mutation that is targeted in the methods and use as disclosed herein is the G>A mutation leading to the change of glutamate to lysine at position 504 of the mature protein. In an embodiment, disclosed is a method of treating alcohol intolerance, or a disorder caused by said alcohol intolerance, the method comprising administering to a patient in need thereof a therapeutically effective amount of an EON as disclosed herein, a vector as disclosed herein, or a pharmaceutical composition as disclosed herein.

In an embodiment, disclosed is a method for the deamination of a target adenosine in an ALDH2 pre-mRNA or mRNA molecule in a cell, the method comprising the steps of: (i) providing the cell with an EON or a vector as disclosed herein; (ii) allowing uptake by the cell of the EON or the vector, respectively; (iii) allowing annealing of the EON to the ALDH2 pre-mRNA or mRNA molecule; (iv) allowing an endogenous ADAR enzyme, such as ADAR2, to deaminate the target adenosine in the target RNA molecule to an inosine; and optionally (v) identifying the presence of the inosine in the target RNA molecule. A preferred target adenosine is a G>A mutation in the transcript molecule of a mutant ALDH2 gene coding for a mutant p.E504K ALDH2 protein. Preferably, step (v) comprises a) determining the sequence of the ALDH2 pre-mRNA or mRNA molecule; b) assessing the presence of a wild-type ALDH2 protein; or c) using a functional read-out, such as assessing a level of alcohol in a serum or plasma sample, or any other biomarker related to the function of ALDH2 known to the person skilled in the art.

### Definitions

The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro(di)thioate, MP, methyl thiophosphonate, phosphoramidate linkages, and the like. Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Thymine (T) is also known as 5-methyluracil (m⁵U) and is a uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an EON as disclosed herein may comprise a mix of ribonucleosides and deoxyribonucleosides. When a deoxyribonucleotide is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, isouracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, linkages between two mononucleotides may be phosphodiester linkages as well as modifications thereof, including, phosphonoacetate, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%.

The word ‘substantially’ does not exclude ‘completely’, e.g., a composition which is ‘substantially free from Y’ may be completely free from Y. Where relevant, the word ‘substantially’ may be omitted from the definition of the invention.

The term ‘complementary’ as used herein refers to the fact that the EON hybridizes under physiological conditions to a second nucleic acid strand (for instance when the oligonucleotide as a first nucleic acid strand (=guide oligonucleotide) forms a heteroduplex RNA editing oligonucleotide complex, or HEON, with another complementary nucleic acid strand), or when it forms a double stranded complex with the target RNA sequence. The term does not necessarily mean that each nucleotide in a nucleic acid strand has a perfect pairing with its opposite nucleotide in the opposite sequence. In other words, while an EON may be complementary to a target sequence, there may be mismatches, wobbles and/or bulges between the oligonucleotide and the target sequence, while under physiological conditions that EON still hybridizes to the target sequence such that the cellular RNA editing enzymes can edit the target adenosine. The term ‘substantially complementary’ therefore also means that despite the presence of the mismatches, wobbles, and/or bulges, the EON has enough matching nucleotides between the EON and target sequence that under physiological conditions the EON hybridizes to the target RNA. As shown herein, an EON may be complementary, but may also comprise one or more mismatches, wobbles and/or bulges with the target sequence, if under physiological conditions the EON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments first nucleic acid strands of the present invention comprise fewer than four mismatches with the target sequence, for example 0, 1 or 2 mismatches. ‘Wobble’ base pairs are G-U, I-U, I-A, and I-C base pairs. Although a G:G pairing would be considered a mismatch, that does not necessarily mean that the interaction is unstable, which means that the term ‘mismatch’ may be somewhat outdated based on the current invention where a Hoogsteen base-pairing may be seen as a mismatch based on the origin of the nucleotide but still be relatively stable. An isolated G:G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

An EON (and the complementary nucleic acid strand when two oligonucleotides form a HEON) as disclosed herein may be chemically modified almost in its entirety, for example by providing nucleotides with a ribose sugar moiety carrying a 2′-OMe substitution, a 2′-F substitution, or a 2′-O-methoxyethyl (2′-MOE) substitution. The orphan nucleotide in the EON is preferably a cytidine or analog thereof (such as a nucleotide carrying a Benner's base), or a uridine or analog thereof (such as iso-uridine), and/or in one embodiment comprises a diF modification at the 2′ position of the sugar, in another embodiment comprises a deoxyribose (2′-H, DNA), and in yet a further embodiment, at least one and in another embodiment both the two neighbouring nucleotides flanking the orphan nucleotide do not comprise a 2′-OMe modification. Complete modification wherein all nucleotides of the oligonucleotide hold a 2′-OMe modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modification are known in the field of oligonucleotides that can be readily used in accordance with the invention. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers.

In an embodiment, the EON of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. Notably, when the EON is delivered through a (viral) vector, the length may increase as being longer than 60 nucleotides. However, when the EON is to be delivered as is, without a vector, also referred to as a ‘naked form’, the length of the EON is limited to 15 to 60 nucleotides to reduce the risk of degradation. Furthermore, in a naked form, the EON is preferably chemically modified as outlined herein to lower the risk of degradation.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably help to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

The RNA editing protein present in the cell that is of most interest to be used with an EON of the present invention is human ADAR2. It will be understood by a person having ordinary skill in the art that the extent to which the editing entities inside the cell are redirected to other target sites may be regulated by varying the affinity of the first nucleic acid strand for the recognition domain of the editing molecule. The exact modification may be determined through some trial and error and/or through computational methods based on structural interactions between the EON and the recognition domain of the editing molecule. In addition, or alternatively, the degree of recruiting and redirecting the editing entity resident in the cell may be regulated by the dosing and the dosing regimen of the EON. This is something to be determined by the experimenter (in vitro) or the clinician, usually in phase I and/or II clinical trials.

The invention concerns the modification of target RNA sequences in eukaryotic, preferably metazoan, more preferably mammalian, even more preferably human cells, and most preferably human liver cells such as hepatocytes. The invention is particularly suitable for modifying RNA sequences in cells and tissues in which ALDH2K is expressed and wherein that protein acts. Because ALDH2 is predominantly produced and has an important role in liver cells in ethanol metabolism, the preferred target cell for the EONs of the present invention are liver cells, more preferably hepatocytes. The target cell can be located in vitro, ex vivo or in vivo. One advantage of the invention is that it can be used with cells in situ in a living organism, but it can also be used with cells in culture. In some embodiments cells are treated ex vivo and are then introduced into a living organism (e.g., re-introduced into an organism from whom they were originally derived). The invention can also be used to edit target RNA sequences in cells from a transplant or within a so-called organoid, e.g., a liver tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through human ADAR2 is thought to take place on primary transcripts in the nucleus, during transcription or splicing, or in the cytoplasm, where e.g., mature mRNA, miRNA or ncRNA can be edited.

It should be clear, that targeted editing according to the invention can be applied to any adenosine within the ALDH2 transcript if the deamination of the adenosine results in an increase or restoration of ALDH2 protein function. As outlined herein, it is however preferred to target the first adenosine that is present in a mutated codon at position 504 of the mature protein.

Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present invention. Hence, any RNA editing of a target adenosine in the ALDH2 transcript and that results in improvement or restoration of the ALDH2 protein function is encompassed by the present invention. The present invention opens a whole new field of treating alcohol intoxication, using genetic editing techniques.

The amount of EON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker. It is possible that higher doses of EONs could compete for binding to an ADAR within a cell, thereby depleting the amount of the entity, which is free to take part in RNA editing, but routine dosing trials will reveal any such effects for a given EON and a given target.

One suitable trial technique involves delivering the EON to cell lines, or a test organism and then taking biopsy samples at various time points thereafter. The sequence of the target RNA can be assessed in the biopsy sample and the proportion of cells having the modification can easily be followed. Also, alcohol percentage in a blood sample is a proper biomarker for assessing the function of the ALDH2 protein in a particular subject, before and after treatment, or with or without treating the subject with an EON or vector as disclosed herein. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, as indicated above, the change may be assessed on the function of the protein, for instance by measuring or assessing a serum or plasma ethanol concentration before, during, and/or after treatment or assessing any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method of the invention may involve repeated delivery of an EON until enough target RNAs have been modified to provide a tangible benefit to the patient and/or to maintain the benefits over time.

EONs of the invention are particularly suitable for therapeutic use, and so the invention also relates to a pharmaceutical composition comprising an EON of the invention, or a vector or plasmid encoding the EON of the invention, and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g., syringe, inhaler, nebuliser) which includes a pharmaceutical composition of the invention.

The invention also provides an EON of the invention for use in a method for repairing a mutation in a target ALDH2 RNA sequence in a mammalian, preferably a human liver cell, as described herein. Similarly, the invention provides the use of an EON of the invention in the manufacture of a medicament for making a change in a target ALDH2 RNA sequence in a mammalian, preferably a human liver cell, as described herein, and thereby treating, preventing, or ameliorating diseases related to alcohol intoxication, such as those caused by ALDH2*2.

The invention also relates to a method for the deamination of at least one specific target adenosine present in a target ALDH2 RNA sequence in a cell, the method comprising the steps of: providing the cell with an EON according to the invention; allowing uptake by the cell of the EON; allowing annealing of the EON to the target RNA molecule; allowing a mammalian ADAR enzyme comprising a natural dsRNA binding domain as found in the wild type enzyme to deaminate the target adenosine (preferably the first adenosine of the codon encoding lysine at position 504 in the mature protein) in the target RNA molecule to an inosine; and optionally identifying the presence of the inosine in the RNA sequence.

The invention also relates to a method for the deamination of at least one specific target adenosine present in a target ALDH2 RNA sequence in a cell, the method comprising the steps of: providing the cell with a vector or plasmid encoding the EON according to the invention; allowing uptake by the cell of the vector or plasmid; allowing annealing of the EON to the target RNA molecule; allowing a mammalian ADAR enzyme comprising a natural dsRNA binding domain as found in the wild type enzyme to deaminate the target adenosine (preferably the first adenosine of the codon encoding lysine at position 504 in the mature protein) in the target RNA molecule to an inosine; and optionally identifying the presence of the inosine in the RNA sequence.

In a preferred aspect, depending on the ultimate deamination effect of A to I conversion, the identification step comprises the following steps: sequencing the target RNA; assessing the presence or absence of a functional protein; assessing whether splicing of the pre-mRNA was altered by the deamination; or using a functional read-out, because the target RNA after the deamination should encode a functional protein. Examples are assessing ethanol and/or acetaldehyde concentrations in (blood) samples after RNA editing. The identification of the deamination into inosine may therefore be a functional read-out using a suitable biomarker. The functional assessment for alcohol intoxication mentioned herein will generally be according to methods known to the skilled person. A very suitable manner to identify the presence of an inosine after deamination of the target adenosine is of course dPCR or even sequencing, using methods that are well-known to the person skilled in the art. However, the person skilled in the art of liver disease may apply tests to monitor certain biomarkers related to alcohol intoxication, as discussed above.

The EON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one embodiment, a method according to the invention comprises the steps of administering to the subject an EON or pharmaceutical composition according to the invention, allowing the formation of a double stranded nucleic acid complex of the EON with its specific complementary target nucleic acid molecule in a cell in the subject; allowing the engagement of an endogenous present adenosine deaminating enzyme, such as ADAR2; and allowing the enzyme to deaminate the target adenosine in the target nucleic target molecule to an inosine, thereby alleviating, preventing or ameliorating the disease related to alcohol intoxication. The diseases that may be treated according to this method are preferably, but not limited to, the genetic diseases listed herein, and any other disease in which deamination of an adenosine in ALDH2 transcripts would restore the protein's function in a patient in need thereof.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 100 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and EONs according to the invention are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### Chemical Modifications

All chemical modifications listed below that may be used in the EON of the present invention may also be used for a sense strand that is complementary to the EON, when the EON and the complementary strand form a so-called heteroduplex RNA editing oligonucleotide (HEON) complex, as described in GB 2215614.5 (unpublished), except that the opposite sense strand does not have an orphan nucleotide. Hence, the modification related to the orphan nucleotide relate only to the EON of the present invention, but all other modifications relate to the EON of the present invention and any (protecting) sense oligonucleotide that may be used together with the EON in a pharmaceutical product. This includes the use of hydrophobic moieties (such as tocopherol and cholesterol) and cell-specific ligands (such as GalNAc moieties), that have also been described herein, and in detail in GB 2215614.5 (unpublished), which may either be bound to the EON or its opposite strand, or both.

The internucleoside linkages in the oligonucleotides of the present invention may comprise one or more naturally occurring internucleoside linkages and/or modified internucleoside linkages. Without limitations, at least one, at least two, or at least three internucleoside linkages from a 5′ and/or 3′ end of the EON are preferably modified internucleoside linkages. A preferred modified internucleoside linkage is a PS linkage. In one embodiment, all internucleoside linkages of the EON are modified internucleoside linkages. In one embodiment, the EON comprises a PNdmi linkage linking the most terminal nucleoside at the 5′ and/or 3′ end, and the one before last nucleoside at each of these ends, respectively. A PNdmi linkage as preferably used in the EONs of the present invention has the structure of the following formula:

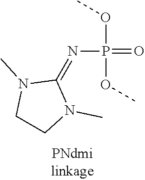

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (often abbreviated to 2′-OMe or 2′-O-Me), 2′-F and 2′-O-methoxyethyl (often also referred to as 2′-methoxyethoxy, or 2′-MOE) modifications of the sugar and the use of PS linkages between nucleosides. WO2020/201406 discloses the use of MP linkage modifications at certain positions surrounding the orphan nucleotide in the first nucleic acid strand. The ribose 2′ groups in all nucleotides of the EON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the EON that comprises no other chemical modifications to the ribose sugar, the base, or the linkage preferably does not carry a 2′-OMe or 2′-MOE substitution but may carry a 2′-F, a 2′,2′-difluoro (diF), or 2′-ara-F (FANA) substitution or may be DNA. GB 2214347.3 (unpublished) describes the modification of the 2′ position of the ribose sugar moiety of the orphan nucleotide by a 2′,2′-disubstituted substitution such as diF, which is also applicable to the invention described here. The 2′-4′ linkage can be selected from many linkers known in the art, such as a methylene linker, amide linker, or constrained ethyl linker (cEt).

The invention relates to an EON for use in the deamination of a target nucleotide (preferably adenosine) in a target RNA, wherein the EON is complementary to a stretch of nucleotides in the target RNA that includes the target adenosine, wherein the nucleotide in the first nucleic acid strand that is directly opposite the target nucleotide is the orphan nucleotide, and when the target nucleotide is an adenosine the orphan nucleotide comprises preferably a base or modified base or base analogue with a NH moiety at the position similar to the ring nitrogen (e.g., Benner's base Z). The nucleotide numbering in the EON is such that the orphan nucleotide is number 0 and the nucleotide 5′ from the orphan nucleotide is number +1. Counting is further positively (+) incremented towards the 5′ end and negatively (−) incremented towards the 3′ end, wherein the first nucleotide 3′ from the orphan nucleotide is number −1. The internucleoside linkage numbering in the EON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the EON comprises one or more (chirally pure or chirally mixed) PS linkages. In one embodiment, the PS linkages connect the terminal 3, 4, 5, 6, 7, or 8 nucleotides on each end of the first nucleic acid strand. In one embodiment, the EON comprises one of more phosphoramidate (PN) linkages. In one embodiment, a PN linkage connects the terminal two nucleotides on each end of the EON.

A nucleoside in the EON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-0-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl](MOCE), 2′-O-[2-N-methylcarbamoyl)ethyl](MCE), 2′-O-[2-(N,N-dimethylcarbamoyl)ethyl](DCME); 2′-halo e.g. 2′-F, FANA; 2′-O-[2-(methylamino)-2-oxoethyl](NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The base sequence of the EON herein is complementary to part of the base sequence of a target ALDH2 transcription product that includes at least the target adenosine that is to be deaminated to an inosine, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

The EON according to the present invention, in contrast to what has been described for gapmers and their relation towards RNase breakdown and the use of such gapmers in double-stranded complexes (see for instance EP 3954395 A1), does not comprise a stretch of DNA nucleotides which would make a target sequence (or a sense nucleic acid strand) a target for RNase-mediated breakdown. In one embodiment, the EON does not comprise four or more consecutive DNA nucleotides anywhere within its sequence. In an embodiment, the EON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the EON may be DNA, but also that there is no stretch of four or more consecutive DNA nucleotides within the EON. Hence, the EON according to the present invention is not a gapmer. A gapmer reduces the expression of a target transcript but does not produce RNA editing of a specified adenosine within the target transcript. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the EON according to the invention may be any oligonucleotide that produces an RNA editing effect in which a target adenosine in a target RNA molecule is deaminated to an inosine, and accordingly is resistant to RNase-mediated breakdown as much as possible to yield this effect.

In one embodiment, the EON, or the sense strand to which it may be annealed before entering a target cell, is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, TEG, HEG, alkyl chains, propyl, 6-aminohexyl, or dodecyl.

The invention also relates to a pharmaceutical composition comprising the EON according to the invention, and further comprising a pharmaceutically acceptable carrier and/or other additive and may be dissolved in a pharmaceutically acceptable organic solvent, or the like. Dosage forms in which the EON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

In one embodiment, the EON comprises at least one nucleotide with a sugar moiety that comprises a 2′-OMe modification. In one embodiment, the EON comprises at least one nucleotide with a sugar moiety that comprises a 2′-MOE modification. In one embodiment, the EON comprises at least one nucleotide with a sugar moiety that comprises a 2′-F modification. In one embodiment, the orphan nucleotide carries a 2′-H in the sugar moiety and is therefore referred to as a DNA nucleotide, even though additional modifications may exist in its base and/or linkage to its neighbouring nucleosides. In one embodiment, the orphan nucleotide carries a 2′-F in the sugar moiety. In one embodiment, the orphan nucleotide carries a diF substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the EON is an antisense oligonucleotide that can form a double stranded nucleic acid complex with a target RNA molecule, wherein the double stranded nucleic acid complex can recruit an adenosine deaminating enzyme for deamination of a target adenosine in the target ALDH2 RNA molecule, wherein the nucleotide in the EON that is opposite the target adenosine is the orphan nucleotide, and wherein the orphan nucleotide has the following structure:

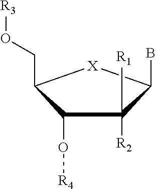

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides. The nucleotide 3′ and/or 5′ from the orphan nucleotide may be DNA, more preferably the nucleotide at the 3′ (position −1).

In one embodiment, the first nucleic acid strand comprises at least one MP internucleoside linkage according to the following structure:

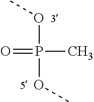

A preferred position for an MP linkage in an EON according to the invention is linkage position −1, thereby connecting the nucleoside at position −1 with the nucleoside at position −2, although other positions for MP linkages are not explicitly excluded.

In one embodiment, the EON comprises at least one nucleotide with a sugar moiety that comprises a 2′-fluoro (2′-F) modification. A preferred position for the nucleotide that carries a 2′-F modification is position −3 in EON, which may be present together with an identical 2′ modification in the orphan nucleotide as discussed above.

In one embodiment, the EON comprises at least one phosphonoacetate or phosphonoacetamide internucleoside linkage.

In one embodiment, the EON comprises at least one nucleotide comprising a locked nucleic acid (LNA) ribose modification, or an unlocked nucleic acid (UNA) ribose modification. In an embodiment, the EON comprises at least one nucleotide comprising a threose nucleic acid (TNA) ribose modification.

The skilled person knows that an oligonucleotide, such as an EON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the EON of the present invention are disclosed in WO2020/154342, WO2020/154343, and WO2020/154344.

In one embodiment, the EON of the present invention may comprise one or more nucleotides carrying a 2′-MOE ribose modification. Also, in one embodiment, the EON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the EON comprises 2′-OMe ribose modifications at the positions that do not comprise a 2′-MOE ribose modification, and/or wherein the oligonucleotide comprises deoxynucleotides at positions that do not comprise a 2′-MOE ribose modification. In one embodiment the EON comprises one or more nucleotides comprising a 2′ position comprising a 2′-MOE, 2′-OMe, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g.

WO2018/007475)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the EON fulfils its role as an editing producing oligonucleotide that can form a double stranded complex with the target RNA and recruit a deaminating enzyme, that can subsequently deaminate the target adenosine. Where a monomer comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar.

The nucleobases in an EON of the present invention can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine, or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

EONs according to the invention can comprise linkage modifications. A linkage modification can be, but not limited to, a modified version of the phosphodiester present in RNA, such as PS, chirally pure PS, (R)-PS, (S)-PS, methyl phosphonate (MP), chirally pure methyl phosphonate, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′43′ and 2′45′ linkages.

In one embodiment, an EON comprises a substitution of one of the non-bridging oxygens in the phosphodiester linkage. This modification slightly destabilizes base-pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Many of these non-naturally occurring modification of the linkage, such as PS are chiral, which means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of providing RNA editing. However, if such is not specifically desired, a composition may comprise AONs as active compounds with both Rp and Sp configurations at a certain specified linkage position. Mixtures of such EONs are also feasible, wherein certain positions have preferably either one of the configurations, while for other positions such does not matter.

Again, in all cases, the modifications should be compatible with editing such that the EON fulfils its role as an editing producing oligonucleotide that can, when attached to its target sequence recruit an adenosine deaminase enzyme because of the dsRNA nature that arises. In all aspects of the invention, the enzyme with adenosine deaminase activity is preferably ADAR1, ADAR2, or ADAT. In a highly preferred embodiment, the EON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The invention also relates to a pharmaceutical composition comprising the EON as characterized herein, and a pharmaceutically acceptable carrier.

Other chemical modifications of the EON according to the invention include the substitution of one or more than one of any of the hydrogen atoms with deuterium or tritium, examples of which can be found in e.g., WO2014/022566 or WO2015/011694.

The invention relates to an EON according to the invention, or a pharmaceutical composition comprising an EON according to the invention, for use in the treatment or prevention of a disorder related to alcohol intolerance, or alcohol intoxication, preferably caused by the ALDH2*2 mutant. In one embodiment, the invention relates to an EON according to the invention, or a pharmaceutical composition comprising an EON according to the invention, for use in the treatment or prevention of a disease related to alcohol intolerance, or alcohol intoxication, preferably caused by the ALDH2*2 mutant. In one embodiment, the invention relates to an EON according to the invention, or a pharmaceutical composition comprising an EON according to the invention, for use in the treatment or prevention of alcohol intolerance, or alcohol intoxication, preferably caused by the ALDH2*2 mutant.

EONs of the present invention preferably do not include a 5′-terminal O6-benzylguanosine or a 5′-terminal amino modification and preferably are not covalently linked to a SNAP-tag domain (an engineered O6-alkylguanosine-DNA-alkyl transferase). EONs of the present invention preferably do not comprise a boxB RNA hairpin sequence. In one embodiment, an EON of the present invention comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is uridine. One alternative for uridine is positioning an iso-uridine opposite the target adenosine, which likely does not pair like G pairs with U. Preferably, the target adenosine in the target sequence forms a mismatch base pair with the nucleoside in the EON that is directly opposite the target adenosine.

It should be noted that when an EON is delivered through a vector, for instance an AAV vector, chemical modifications are not present in the EON that acts on the target RNA molecule. Although it is preferred to use ‘naked’ EONs that have chemical modifications as outlined herein, EONs that are delivered through other means, for instance through AAV vector expression, or editing molecules that are circular, or have hairpin structures (recruiting portions, e.g., as disclosed in WO2016/097212, WO2017/050306, WO2020/001793, WO2017/010556, WO2020/246560, and WO2022/078995) are also encompassed by the present invention because these can also be applied to edit adenosines in the target ALDH2 RNA molecule to generate a ALDH2 protein with restored function.

An EON according to the present invention can utilise endogenous cellular pathways and naturally available ADAR enzymes to specifically edit a target adenosine in the target RNA sequence. An EON of the invention is capable of recruiting ADAR and complex with it and then facilitates the deamination of a (single) specific target adenosine nucleotide in a target RNA sequence. Ideally, only one adenosine is deaminated. An EON of the invention, when complexed to ADAR, preferably brings about the deamination of a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the EONs and the target RNA also appears important to the development of efficient ADAR-based EON therapy.

As outlined above, an EON of the present invention makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the phosphodiester linkages, as outlined in detail herein. They may also be variably distributed throughout the sequence of the EON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the EON, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. The invention relates to RNA editing oligonucleotides, generally referred to as EONs herein, that can bring about deamination of an adenosine in the ALDH2 transcript, with a resulting ALDH2 protein that is fully functional in ethanol metabolism. This means that the invention is not strictly limited to deamination of the adenosine of mutant ALDH2*2, but that other (single or multiple) adenosines may be targeted, which may also result in increased ALDH2 protein function. Other adenosines may be identified, for instance by genetic screening in the population, or in silico, that are also important (or may become more important) for ALDH2 function, and that also may be targeted through RNA editing, following the teaching of the present invention. All such RNA events and oligonucleotides that can be used for such targeting are encompassed by the present invention, no matter what the exact nucleic molecule, or EON, looks like.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. Proc Natl Acad Sci USA 2012. 109(48):3295-3304). During the deamination reaction, ADAR flips the edited base out of its RNA duplex, and into the enzyme active site (Matthews et al. 2016). When ADAR2 edits adenosines in the preferred context (an A:C mismatch) the nucleotide opposite the target adenosine is often referred to as the ‘orphan cytidine’. The crystal structure of ADAR2 E488Q bound to double stranded RNA (dsRNA) revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of EONs with modified RNA bases, especially at the position of the orphan cytidine to mimic the hydrogen-bonding pattern observed by the E488Q ADAR2 mutant. By replacing the nucleotide opposite the target adenosine in the EON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74(21):8021-8030; Burchenal et al. (1976) Cancer Res 36:1520-1523) and Benner's base Z (also referred to as ‘dZ’; Yang et al. Nucl Acid Res 2006. 34(21):6095-6101) that were initially selected because they offer hydrogen-bond donation at N3 with minimal perturbation to the shape of the nucleobase. Benner's base is also referred to as 6-amino-5-nitro-2(1H)-pyridone. The presence of the cytidine analog in the AON may exist in addition to modifications to the ribose 2′ group. The ribose 2′ groups in the AON can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

In one embodiment, a nucleotide analogue or equivalent within the EON comprises one or more base modifications or substitutions. Modified bases comprise synthetic and natural bases such as inosine, xanthine, hypoxanthine and other-aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An EON according to the invention is normally longer than 10 nucleotides, preferably more than 11, 12, 13, 14, 15, 16, still more preferably more than 17 nucleotides. In one aspect the AON according to the invention is longer than 20 nucleotides. The oligonucleotide according to the invention is preferably shorter than 100 nucleotides, still more preferably shorter than 60 nucleotides, still more preferably shorter than 50 nucleotides. In a preferred aspect, the oligonucleotide according to the invention comprises 18 to 70 nucleotides, more preferably comprises 18 to 60 nucleotides, and even more preferably comprises 18 to 50 nucleotides. Hence, in a particularly preferred aspect, the oligonucleotide of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In one embodiment, the AON is 27, 28, 29, or 30 nucleotides in length.

In one aspect, at either end or both termini of an EON according to the present invention inverted deoxyT or dideoxyT nucleotides are incorporated.

As described above, in some embodiments the invention provides an EON for forming a double stranded complex with a human ALDH2 RNA molecule in a human liver cell. Thus, the therapeutic effect is preferably on a human liver cell in vivo. Of course, the methods may also be carried out in vitro or ex vivo.

The invention provides an EON of the invention, or pharmaceutical composition of the invention, for use in the treatment of disease. The invention also provides the use of an EON of the invention, or pharmaceutical composition of the invention, in the manufacture of a medicament for the treatment of disease. The invention also provides a method for treating a disease in a patient, comprising administering a therapeutically effective amount of an EON according to the invention or a pharmaceutical composition according to the invention. Preferably the disease is a disease caused by the E504K mutation in ALDH2. The EON is administered therapeutically or prophylactically (after genetic counselling) because both types of treatment could be beneficial.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

### EXAMPLES

### Example 1. Editing of a Target Adenosine in a Human ALDH2 Target RNA Molecule Using an In Vitro Biochemical Editing Assay

First, an initial set of the ALDH2-targeting EONs (shown in FIG. 1) are tested to address editing of human ALDH2 target (pre-) mRNA in an in vitro biochemical editing assay. To obtain the ALDH2 target RNA a PCR is performed using an ALDH2 G-block (IDT) which contains the sequence for the T7 promotor and (a part of) the sequence of ALDH2 as template using forward primer 5′-CTC GAC GCA AGC CAT AAC AC-3′ (SEQ ID NO:53) and reverse primer 5′-TGG ACC GAC TGG AAA CGT AG-3′ (SEQ ID NO:54). The 5′ to 3′ G-block sequence (SEQ ID NO:55) is as follows, in which the target adenosine is underlined and in bold, and in which the primer sequences are underlined:

TCTGGCTCGACGCAAGCCATAACACTAATACGACTCACTATAGGG
GGAGCCCAGTCACCCTTTGGTGGCTACAAGATGTCGGGGAGTGGC
CGGGAGTTGGGCGAGTACGGGCTGCAGGCATACACTAAAGTGAAA
ACTGTCACAGTCAAAGTGCCTCAGAAGAACTCATAAGAATCATGC
AAGCTTCCTCCCTCAGCCATTGATGGAAAGTTCAGCAAGATCAGC
AACAAAACCAAGAAAAATGATCCTTACGTTTCCAGTCGGTCCACG
TTTG

The PCR product is then used as template for the in vitro transcription. The MEGAscript T7 transcription kit is used for this reaction. The RNA is purified on a urea gel and then extracted in 50 mM Tris-Cl pH 7.4, 10 mM EDTA, 0.1% SDS, 0.3 M NaCl buffer and subsequently phenol-chloroform purified. The purified RNA is used as target in the biochemical editing assay.

Initially, EONs ALDH2-01, 02, 05, 06, 09, 10, 13, 14, 17, 18, 21, 22, 25, 26, 29, 30, 33, 34, 37, 38, 41, 42, 45, and 46 are annealed to the ALDH2 target RNA, which is done in a buffer (5 mM Tris-Cl pH 7.4, 0.5 mM EDTA and 10 mM NaCl) at the ratio 1:3 of target RNA to oligonucleotide (600 nM oligonucleotide and 200 nM target). The samples are heated at 95° C. for 3 min and then slowly cooled down to RT. Next, the editing reaction is carried out. The annealed oligonucleotide/target RNA is mixed with protease inhibitor (cOmplete™, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT) such that their final concentration is 6 nM oligonucleotide and 2 nM target RNA. The reaction is started by adding purified ADAR2 (GenScript) to a final concentration of 6 nM into the mix and incubated for predetermined time points at 37° C. Each reaction is stopped by adding 95 μl of 95° C. 3 mM EDTA solution. A 6 μl aliquot of the stopped reaction mixture is then used as template for cDNA synthesis using Maxima reverse transcriptase kit (Thermo Fisher) with random hexamer primer (ThermoFisher Scientific). Initial denaturation of RNA is performed in the presence of the primer and dNTPs at 95° C. for 5 min, followed by slow cooling to 10° C., after which first strand synthesis is carried out according to the manufacturer's instructions in a total volume of 20 μl, using an extension temperature of 62° C. Products are amplified for pyrosequencing analysis by PCR, using the Amplitaq gold 360 DNA Polymerase kit (Applied Biosystems) according to the manufacturer's instructions, with 1 μl of the cDNA as template. Then PCR is performed using the following thermal cycling protocol: Initial denaturation at 95° C. for 5 min, followed by 40 cycles of 95° C. for 30 sec, 58° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of guanosine (edited) versus adenosine (unedited) is defined by pyrosequencing. Pyrosequencing of the PCR products and data analysis is performed by the PyroMark Q48 Autoprep instrument (QIAGEN) following the manufacturer's instructions with 10 μl input of the PCR product and 4 μM sequencing primer: The analysis performed by the instrument provides the results for the selected nucleotide as a percentage of adenosine and guanosine detected in that position, and the extent of A-to-I editing at a chosen position is therefore measured by the percentage of guanosine in that position.

### Example 2. Editing of a Target Adenosine in a Human ALDH2 Target RNA Molecule Using Patient Derived Skin Fibroblasts

It was investigated whether the modified EONs shown in FIG. 1, each carrying an RM number, would be able to edit the target adenosine in human ALDH2*2 transcript RNA carrying the c.1510G>A mutation, in cells. For this, untransformed human skin fibroblasts (AG11369; Cornell, USA) were used that are heterozygous for the c.1510G>A mutation. The disadvantage of using heterozygous mutated cells is that there is a significant background signal of non-edited transcripts, which are wild type and are transcribed from the wild type allele. About 50,000 cells per 24 well plate, were seeded 24 hrs before administration of the respective EONs, which was either performed with 100 nM EON alone, or in the context of 5 μM saponin (AG1856; see WO2021/122998). Cells were incubated with EONs±saponin for 3 days before harvesting. RNA was extracted using the Direct-zol RNA MicroPrep (Zymo Research) kit according to the manufacturer's instructions, and cDNA was prepared using the Maxima reverse transcriptase kit (ThermoFisher) according to the manufacturer's instructions, with random hexamers. The cDNA was used as template for digital PCR (dPCR) with respectively 200 ng RNA input per reaction. The dPCR assay for absolute quantification of nucleic acid target sequences was performed using Qiagen's QIAcuity 4 Digital PCR system. 1.2 μl of undiluted cDNA obtained from the RT cDNA synthesis reaction was used in a total mixture of 12 μl of reaction mix, including the 4× concentrated QIAcuity Probe Mastermix (Qiagen), a Taqman SNP genotype assay with the following forward and reverse primers combined with the following gene-specific probes:

Forward primer:
(SEQ ID NO: 56)
5′-TGGTGGCTACAAGATGTCGG-3′
Reverse primer:
(SEQ ID NO: 57)
5′-TTATGAGTTCTTCTGAGGCACT-3′
Wild type probe (FAM NFQ labeled):
(SEQ ID NO: 58)
5′-/56-FAM/A+CAGTT+TTCACTT+C+A+GTGT
ATGCC/3IABKFQ/-3′
Mutant probe (HEX NFQ labeled):
(SEQ ID NO: 59)
5′-/5HEX/A+CAGTT+TTCACTT+T+A+GTGTAT
GCCC/3IABKFQ/-3′

A total volume of 12 μl PCR mix including cDNA was filled in the QIAcuity Nanoplate (Qiagen) using a multichannel pipette. After sealing the plate with a Nanoplate seal it was placed into the QIAcuity 4 dPCR device wherein partitioning, PCR amplification and fluorescence measurement was done fully automated. The PCR program was as follows: 1 cycle of enzyme activation for 2 min at 95° C., 40 cycles denaturation for 15 sec at 95° C. and annealing/extension for 30 min at 60° C. After PCR, the plate was imaged and analysed within the QIAcuity 4.

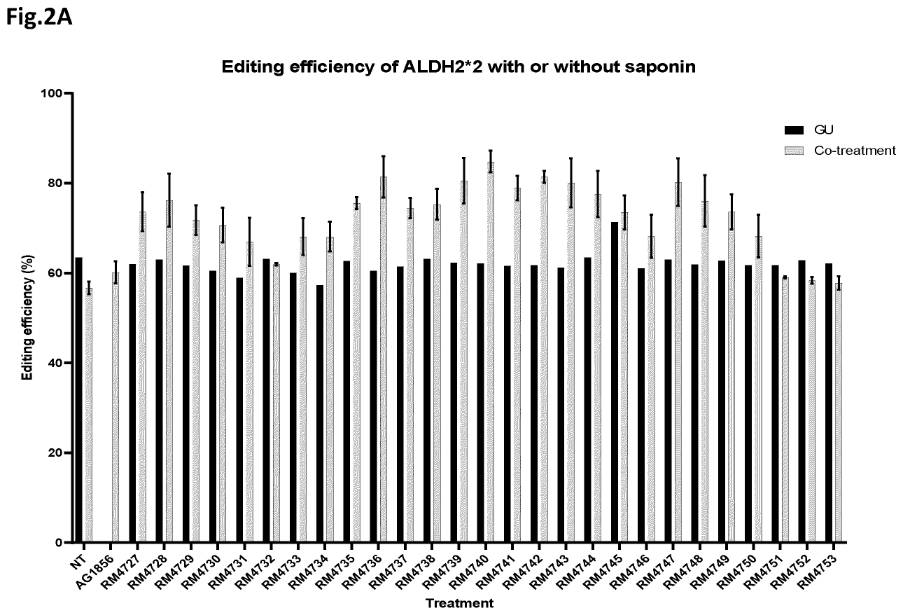

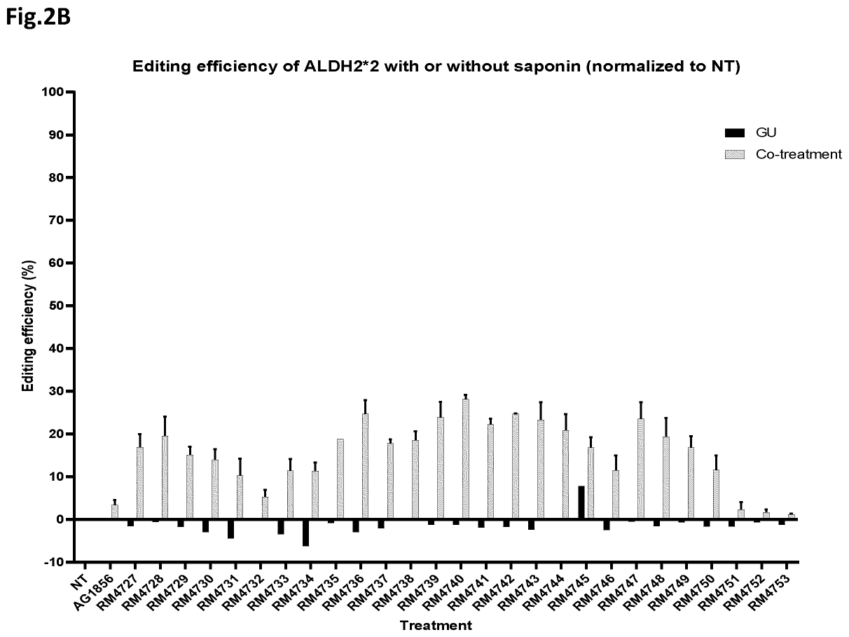

The results given in FIG. 2 show that editing percentages observed in the absence of saponin (gymnotic uptake, or ‘GU’) were similar for most EONs tested, and that the percentage of wild type ALDH2 transcripts reached levels above 80%. However, as indicated above, these values were influenced by the presence of the wild type allele in the heterozygous cells that were used. Normalization to the non-treated sample (NT), which ‘removes’ the percentage of the wildtype background signal, showed that editing levels reached almost 30% when the EONs were administered to the cells in the presence of AG1856, showing that the inventors could edit the ALDH2*2 c.1510G>A mutation to wildtype in human cells, with EON RM4740 performing best.

1. An RNA editing oligonucleotide (EON) capable of forming a double-stranded complex with a region of an endogenous human ALDH2 transcript molecule in a cell, wherein the region of the ALDH2 transcript molecule comprises a target adenosine, and wherein the double-stranded complex can recruit an endogenous ADAR enzyme to deaminate the target adenosine into an inosine, thereby editing the ALDH2 transcript molecule.

2. An EON according to claim 1, wherein the ALDH2 transcript molecule is a pre-mRNA or an mRNA molecule.

3. An EON according to claim 1 or 2, wherein the cell is a human liver cell, preferably a hepatocyte.

4. An EON according to any one of claims 1 to 3, wherein the endogenous ADAR enzyme is ADAR2.

5. An EON according to any one of claims 1 to 4, wherein the target adenosine is the c.1510G>A mutation in the ALDH2 transcript.

6. An EON according to any one of claims 1 to 5, wherein the EON comprises or consists of the sequence of any one of the EON sequences selected from the group consisting of SEQ ID NO:1 to 51.

7. An EON according to any one of claims 1 to 6, wherein at least one nucleotide comprises one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution.

8. An EON according to claim 7, wherein the one or more additional modifications in the linkage moiety is each independently selected from a phosphorothioate (PS), phosphonoacetate, phosphorodithioate, methylphosphonate (MP), sulfonylphosphoramidate, or PNdmi internucleotide linkage.

9. An EON according to claim 7 or 8, wherein the one or more additional modifications in the ribose moiety is a mono- or di-substitution at the 2′, 3′ and/or 5′ position of the ribose, each independently selected from the group consisting of:
-OH;
-F;
substituted or unsubstituted, linear or branched lower (C₁-C₁₀) alkyl, alkenyl, alkynyl, alkaryl, allyl, or aralkyl, that may be interrupted by one or more heteroatoms;
—O-, S-, or N-alkyl;
—O-, S-, or N-alkenyl;
—O-, S-, or N-alkynyl;
—O-, S-, or N-allyl;
—O-alkyl-O-alkyl;
-methoxy;
-aminopropoxy;
-methoxyethoxy;
-dimethylamino oxyethoxy; and
-dimethylaminoethoxyethoxy.

10. A vector, preferably a viral vector, more preferably an adeno-associated virus (AAV) vector, comprising a nucleic acid molecule encoding an EON according to any one of claims 1 to 6.

11. A pharmaceutical composition comprising an EON according to any one of claims 1 to 9, or a vector according to claim 10, and a pharmaceutically acceptable carrier.

12. An EON according to claim 1 to 9, a vector according to claim 10, or a pharmaceutical composition according to claim 11, for use in the treatment of a disorder caused by an ALDH2 deficiency, preferably caused by ALDH2*2.

13. Use of an EON according to any one of claims 1 to 9, or a vector according to claim 10 in the manufacture of a medicament for the treatment of a disorder caused by an ALDH2 deficiency, preferably caused by ALDH2*2, more preferably alcohol intoxication, alcohol poisoning, or symptoms of alcohol consumption.

14. A method of editing a ALDH2 polynucleotide, the method comprising contacting the ALDH2 polynucleotide with an EON capable of effecting an adenosine deaminase acting on RNA (ADAR)-mediated adenosine to inosine alteration of an adenosine associated with alcohol intolerance, thereby editing the ALDH2 polynucleotide.

15. A method of treating a disorder caused by an ALDH2 deficiency, preferably caused by ALDH2*2, in a patient in need thereof, the method comprising contacting a ALDH2 polynucleotide in a cell of the subject with an EON capable of effecting an ADAR-mediated adenosine to inosine alteration of an adenosine associated with the ALDH2 deficiency, thereby treating the patient.

16. A method of treating a disorder caused by ALDH2*2, the method comprising administering to a patient in need thereof a therapeutically effective amount of an EON according to any one of claims 1 to 9, a vector according to claim 10, or a pharmaceutical composition according to claim 11.

17. A method for the deamination of a target adenosine in an ALDH2 pre-mRNA or mRNA molecule in a cell, the method comprising the steps of:
(i) providing the cell with an EON according to any one of claims 1 to 9;
(ii) allowing uptake by the cell of the EON;
(iii) allowing annealing of the EON to the ALDH2 pre-mRNA or mRNA molecule;
(iv) allowing an endogenous ADAR enzyme to deaminate the target adenosine in the target RNA molecule to an inosine; and optionally
(v) identifying the presence of the inosine in the target RNA molecule.

18. A method according to claim 17, wherein the target adenosine is the c.1510G>A mutation in the ALDH2 transcript.

19. A method according to claim 17 or 18, wherein step (v) comprises:
a) determining the sequence of the ALDH2 pre-mRNA or mRNA molecule;
b) assessing the presence of a wild-type ALDH2 protein; or
c) using a functional read-out, preferably assessing a level of alcohol in a serum or plasma sample.
